(19) United States
(12) Patent Application Publication
(10) Pub. No.: US 2017/0164923 A1
(43) Pub. Date: Jun. 15, 2017

(54) Image Processor, Ultrasound Diagnostic Device Including Same, And Image Processing Method

(71) Applicant: KONICA MINOLTA, INC., Chiyoda-ku (JP)

(72) Inventor: Yuki MATSUMOTO, Settsu-shi (JP)

(73) Assignee: KONICA MINOLTA, INC., Tokyo (JP)

(21) Appl. No.: 15/379,085

(22) Filed: Dec. 14, 2016

(30) Foreign Application Priority Data
Dec. 14, 2015 (JP) 2015-243040

(51) Int. Cl. A61B 8/06; A61B 6/00; A61B 6/03; A61B 8/08; A61B 8/00; A61B 5/055; A61B 5/026

(52) U.S. Cl. CPC A61B 8/06; A61B 5/055; A61B 6/507; A61B 5/026; A61B 8/5207; A61B 8/488; A61B 8/463; A61B 6/032; A61B 8/481

(57) ABSTRACT

An image processor including image processing circuitry including: a blood flow image obtainer that obtains a blood flow image in which a blood flow area is mapped, the blood flow area indicating blood flow in the examination subject; a tomographic image obtainer that obtains a tomographic image including an image of a body tissue inside the examination subject; a characteristic obtainer that obtains, based on the blood flow image and the tomographic image, a combination of a first characteristic being a characteristic of the blood flow area itself and a second characteristic indicating a relationship between the blood flow area and the image of a body tissue in the tomographic image; and a blood flow evaluator that evaluates a likelihood of the blood flow area indicating a new blood vessel, based on the combination of the first characteristic and the second characteristic of the blood flow area.

### CROSS-REFERENCE TO RELATED APPLICATION

This application is based on an application No. 2015-243040 filed in Japan, the contents of which are hereby incorporated by reference.

### BACKGROUND OF THE INVENTION

(1) Field of the Invention

The present disclosure relates to an image processor, an ultrasound diagnostic device including the image processor, and an image processing method. In particular, the present invention relates to blood flow-related determination in diagnostic imaging.

(2) Description of the Related Art

Recently, it has become common to conduct diagnostic imaging for joint disorders such as articular rheumatism. Typically, in such diagnostic imaging, areas of blood flow are specified and a determination is made of whether or not new blood vessels formed, for example, through angiogenesis as a result of disorder are present.

For example, the specification of areas of blood flow may be performed by acquiring ultrasound Doppler images by using an ultrasound diagnostic device. Alternatively, the specification of areas of blood flow may be performed by acquiring tomographic images of an examination subject to which an intravascular contrast medium has been administered. In the present disclosure, any image used to specify areas of blood flow, or more specifically, any image showing positions and shapes of areas of blood flow is referred to as a blood flow image.

However, an area of blood flow specified in a blood flow image merely indicates the presence of a blood vessel. Thus, it is significant that in diagnostic imaging for joint disorders, an evaluation is also performed of the likelihood of an area of blood flow specified in a blood flow image indicating a new blood vessel (i.e., the likelihood of an area of blood flow not indicating a pre-existing blood vessel). The term “pre-existing blood vessel” is used in the present disclosure to refer to a blood vessel that is not related to a disorder (e.g., a blood vessel in a healthy body tissue, a blood vessel existing from before the occurrence of disorder).

The evaluation of the likelihood of an area of blood flow in a blood flow image indicating a new blood vessel can be performed, for example, by image-processing the blood flow image and thereby acquiring an index using which the evaluation can be performed quantitively. One example of such method can be found disclosed in Japanese Patent Application Publication No. 2013-144049 (referred to in the following as Patent Literature 1). Specifically, Patent Literature 1 discloses a method of performing filtering, such as noise reduction, on an ultrasound Doppler image and quantitively evaluating the likelihood of an area of blood flow in the ultrasound Doppler image indicating a new-blood vessel based on a relationship between filter characteristics and the amount of change brought about to the ultrasound Doppler image as a result of the filtering. This method makes use of the anatomical viewpoint that new blood vessels and pre-existing blood vessels typically have different structural characteristics (e.g., new blood vessels are usually thinner than pre-existing blood vessels). In addition, the method disclosed in Patent Literature 1 also makes use of the fact that the greater the number of new-blood vessels in an ultrasound Doppler image, the greater the change that even a weak noise reduction filter brings about to the ultrasound Doppler image. That is, an ultrasound Doppler image showing a great number of new blood vessels indicates a greater change with respect to filter characteristics than an ultrasound Doppler image showing a small number of new blood vessels. Based on such facts, Patent Literature 1 determines that an ultrasound Doppler image undergoing much change through filtering as an ultrasound Doppler image showing new blood vessels.

### SUMMARY OF THE INVENTION

### Problems to be Solved by the Invention

However, the method disclosed in Patent Literature 1 performs the evaluation of likelihood of an area of blood flow indicating a new blood vessel by employing only the anatomical viewpoint that new blood vessels are thinner than pre-existing blood vessels, among various anatomical viewpoints that are available. Further, with the method disclosed in Patent Literature 1, any area of blood flow having that is thin is uniformly evaluated as having a high likelihood of indicating a new blood vessel. Due to this, the method disclosed in Patent Literature 1 is not capable of correctly evaluating, for example, a new blood vessel that is not thin or a pre-existing blood vessel that is thin. Thus, in order to correctly evaluate such blood vessels, the method requires a medical practitioner to perform re-evaluation by applying other anatomical viewpoints. In addition, the method disclosed in Patent Literature 1 collectively evaluates an entire ultrasound Doppler image. That is, the method cannot evaluate each individual blood vessel shown in an ultrasound Doppler image showing multiple blood vessels.

In view of the above, the present disclosure provides an image processor, an ultrasound diagnostic device including the image processor, and an image processing method that achieve individually evaluating the likelihood of each area of blood flow shown in a blood flow image indicating a new blood vessel with high reliability, by performing the evaluation by using multiple factors, including but not only the shape of the area.

### Means for Solving the Problems

One aspect of the present disclosure is an image processor including image processing circuitry including: a blood flow image obtainer that obtains a blood flow image in which a blood flow area is mapped, the blood flow area indicating blood flow in an examination subject; a tomographic image obtainer that obtains a tomographic image including an image of a body tissue inside the examination subject; a characteristic obtainer that obtains, based on the obtained blood flow image and the obtained tomographic image, a combination of a first characteristic and a second characteristic of the blood flow area; and a blood flow evaluator that evaluates a likelihood of the blood flow area indicating a new blood vessel, based on the combination of the first characteristic and the second characteristic of the blood flow area, wherein a first characteristic of a blood flow area is defined as being a characteristic of the blood flow area itself, and a second characteristic of a blood flow area is defined as being a characteristic indicating a relationship between the blood flow area and an image of a body tissue in a tomographic image.

### Advantageous Effects of the Invention

The image processor pertaining to one aspect of the present disclosure evaluates the likelihood of a blood flow area in a blood flow image indicating a new blood vessel by using a combination of the first and second characteristic of the blood flow area. The first characteristic is defined as being a characteristic of the blood flow area itself and the second characteristic is defined as being a characteristic indicating a relationship between the blood flow area and an image of a body tissue. Thus, the image processor is capable of reliably evaluating the likelihood of each blood flow area indicating a new blood vessel.

### BRIEF DESCRIPTION OF THE DRAWINGS

These and the other objects, advantages and features of the technology pertaining to the present disclosure will become apparent from the following description thereof taken in conjunction with the accompanying drawings which illustrate a specific embodiment of the technology pertaining to the present disclosure.

FIG. 1 illustrates functional blocks of an image processor 10 pertaining to embodiment 1.

FIG. 2 is a flowchart illustrating operations of the image processor 10.

FIG. 3 is a flowchart illustrating body tissue specification in embodiment 1.

FIG. 4A illustrates an example of a tomographic image in embodiment 1, and FIG. 4B illustrates an example of images of body tissues extracted from the tomographic image.

FIGS. 5A and 5B respectively illustrate an example of a power Doppler image and an example of a color Doppler image, each of which is an example of a blood flow image in embodiment 1.

FIG. 6A illustrates examples of blood flow areas in embodiment 1, and FIGS. 6B and 6C are schematics illustrating examples of relative characteristics of blood flow areas.

FIG. 7A illustrates examples of individual characteristics of blood flow areas, and FIG. 7B illustrates examples of relative characteristics of the blood flow areas.

FIG. 8 is a flowchart illustrating obtaining of relative characteristics in embodiment 1.

FIG. 9 is a flowchart illustrating likelihood evaluation of a blood flow area in embodiment 1.

FIG. 10 is a schematic illustrating the concept of machine learning used for the likelihood evaluation of a blood flow area in embodiment 1.

FIG. 11 illustrates functional blocks of a blood flow evaluator 54 pertaining to a modification.

FIG. 12 is a flowchart illustrating likelihood evaluation of a blood flow area in a modification.

FIG. 13 illustrates functional blocks of an ultrasound diagnostic device 500 pertaining to embodiment 2.

FIG. 14 is a flowchart illustrating operations of the ultrasound diagnostic device 500.

FIGS. 15A, 15B, and 15C are examples of evaluation images in embodiment 2.

### DESCRIPTION OF THE PREFERRED EMBODIMENT

The following provides detailed description of an image processor and an ultrasound diagnostic device including the image processor, each pertaining to one embodiment, with reference to the accompanying drawings.

### Embodiment 1

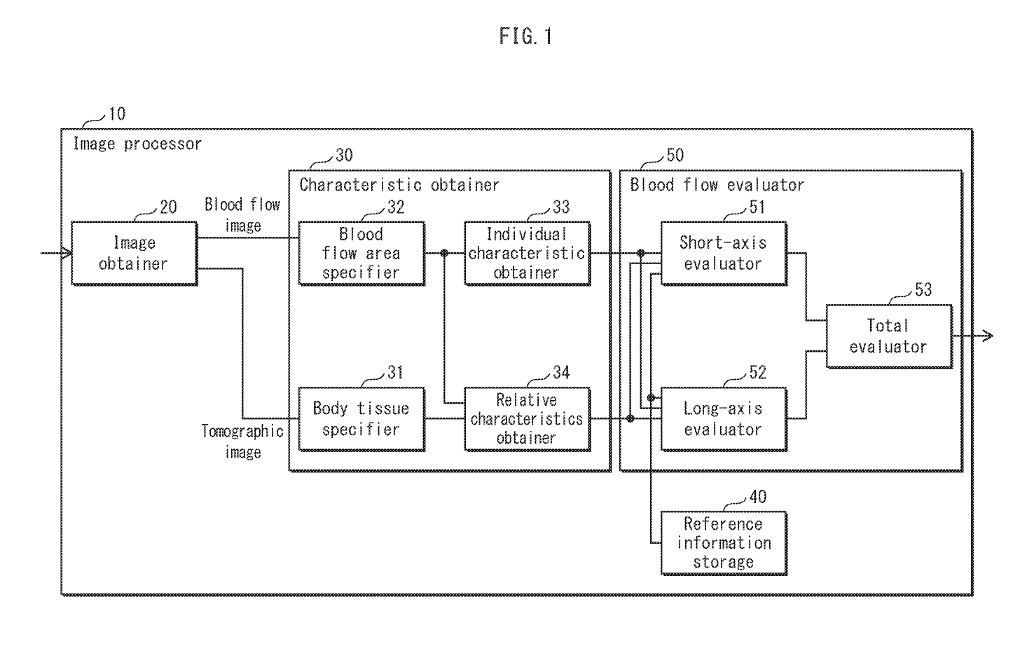

FIG. 1 illustrates functional blocks of an image processor 10 pertaining to embodiment 1. The image processor 10 is an image processing circuit including: an image obtainer 20; a characteristic obtainer 30; a reference information storage 40; and a blood flow evaluator 50.

The image obtainer 20 receives a combination of a blood flow image and a tomographic image. The tomographic image is an image depicting a planar target region set inside the examination subject. Specifically, the tomographic image may be, for example: an ultrasound B-mode image; an X-ray tomographic image such as a computed tomography (CT) image; or a nuclear magnetic resonance image (e.g., a magnetic resonance imaging (MRI) image). The tomographic image includes at least enough information to specify body tissues such as a skin surface and a bone surface. Meanwhile, the blood flow image is an image showing blood flow in the examination subject at the same target region as the tomographic image. The blood flow image may be, for example: an ultrasound Doppler image; an image showing movement (blood flow) inside the examination subject, such as an MRI image; or a contrast medium tomographic image that is a tomographic image captured with a contrast medium administered to the examination subject. The blood flow image includes at least enough information to specify cross-sections of blood vessels at the target region. For example, the information may be an image area indicating movement inside the examination subject or an image of an intravascular contrast medium. Here, it is assumed that the blood flow image and the tomographic images share the same coordinate system. That is, a single point inside the examination subject is indicated by using the same coordinate or the same set of coordinates in the blood flow image and the tomographic image. Due to this, it is preferable that the tomographic image and the blood flow image be obtained at the same timing or at a similar timing. Note that as long as the target region in the blood flow image and the target region in the tomographic image are on the same plane, the blood flow image and the tomographic image need not share the same coordinate system. However, when the blood flow image and the tomographic image have different coordinate systems, information associating the two different coordinate systems with one another becomes necessary. Further, when the blood flow image and the tomographic image have different coordinate systems, the image obtainer 20 uses the information associating the two coordinate systems and performs coordinate conversion on at least one of the blood flow image and the tomographic image so that the coordinate system of the blood flow image and the coordinate system of the tomographic image match. Further, the blood flow image and the tomographic image may be the same image. That is, for example, a single contrast medium tomographic image (e.g., a B-mode image or a CT image) may be used as both the blood flow image and the tomographic image. Further at least one of the blood flow image and the tomographic image may be a frame extracted from a moving image. For example, when both the blood flow image and the tomographic image are extracted from moving images, the image obtainer 20 performs the extraction of frames such that the blood flow image and the tomographic image show the same target region. The image obtainer 20 outputs the blood flow image and the tomographic image to the characteristic obtainer 30.

The characteristic obtainer 30 obtains, for each blood flow area in the blood flow image, a combination of an individual characteristic and a relative characteristic by using the blood flow image and the tomographic image. In the present disclosure, an individual characteristic of a blood flow area is defined as an individual characteristic of the blood flow area (e.g., the shape and the surface area of the blood flow area). Meanwhile, a relative characteristic of a blood flow area is defined as a characteristic indicating a relationship between the blood flow area and one or more body tissues (e.g., the position and the orientation of the blood flow area relative to a body tissue). The characteristic obtainer 30 includes: a body tissue specifier 31; a blood flow area specifier 32; an individual characteristic obtainer 33; and a relative characteristic obtainer 34.

The body tissue specifier 31 extracts images of body tissues, such as a skin surface and a bone surface, from the tomographic image. Specifically, the body tissue specifier 31 first performs edge enhancement on the tomographic image, and then detects edges in the tomographic image. Further, the body tissue specifier 31 specifies what body tissue each edge detected from the tomographic image indicates. For example, the body tissue specifier 31 may perform the edge enhancement by mechanically extracting edges in the tomographic image and overlaying the extracted edges onto the tomographic image. For example, the body tissue specifier 31 may perform the mechanical extraction of edges by using the Sobel filter (Sobel operator), the second derivative, binary processing, the Laplacian filter, or the Canny algorithm. For example, the body tissue specifier 31 may detect edges in the tomographic image by performing dynamic programming. Further, the body tissue specifier 31 performs the specification of what body tissue each edge detected from the tomographic image indicates based on an anatomical viewpoint, or more specifically, based on the relationship between positions of the detected edges in a depth direction of the examination subject. The body tissue specifier 31 outputs information indicating specified body tissues to the relative characteristic obtainer 34.

The blood flow area specifier 32 specifies blood flow areas in the blood flow image. For example, when the blood flow image is an ultrasound Doppler image such as a power Doppler image or a color Doppler image, the blood flow area specifier 32 specifies every area of blood flow in the blood flow image as a blood flow area, irrespective of blood flow velocity, blood flow direction, and blood flow power. A power Doppler image shows blood flow power with color, and shows areas of blood flow in colors indicating blood flow power at the areas. Meanwhile, a color Doppler image shows blood flow direction (i.e., whether blood flow is that towards the ultrasound probe or that away from the ultrasound probe) with color hue and shows blood flow velocity with color brightness, and shows areas of blood flow with hues/brightness levels indicating blood flow direction and blood flow velocity at the areas. This mean that regardless of whether the blood flow image is a power Doppler image or a color Doppler image, a colored area in the blood flow image indicates the presence of blood flow at the area, whereas an uncolored area in the blood flow area indicates the absence of blood flow at the area. Based on this, the blood flow area specifier 32 extracts colored areas of the blood flow image, irrespective of color, and specifies each of the extracted areas as a blood flow area. Alternatively, when the blood flow image is for example a contrast medium tomographic image, the blood flow area specifier 32 may extract dark contrast medium images from the blood flow image, and specify each of such contrast medium images as a blood flow area. Further, when the blood flow image is a contrast medium tomographic image obtained by using ultrasound, the blood flow area specifier 32 may extract contrast medium images from the blood flow image by extracting harmonic components of ultrasound in the contrast medium tomographic image, based on the fact that contrast medium images correspond to harmonic components of ultrasound. The blood flow area specifier 32 outputs information of each blood flow area specified in the blood flow image to the individual characteristic obtainer 33 and the relative characteristic obtainer 34.

The individual characteristic obtainer 33 obtains, for each blood flow area specified by the blood flow area specifier 32, an individual characteristic of the blood flow area. Specifically, the individual characteristic obtainer 33 obtains, as an individual characteristic of a blood flow area, for example, characteristics such as a surface area of the blood flow area and a width of the blood flow area. The individual characteristic obtainer 33 outputs the individual characteristics of the blood flow areas to the blood flow evaluator 50.

The relative characteristic obtainer 34 obtains, for each blood flow area specified by the blood flow area specifier 32, a relative characteristic of the blood flow area. As already described above, a relative characteristic of a blood flow area indicates a relationship between the blood flow area and one or more body tissues specified by the body tissue specifier 31. Specifically, the relative characteristic obtainer 34 obtains, as a relative characteristic of a blood flow area, characteristics such as a horizontal-direction distance of the blood flow area from a center of a joint and a shape similarity between the blood flow area and a bone surface/skin surface. Here, the relative characteristic obtainer 34 obtains relative characteristics of blood flow areas relative to certain body tissues that the relative characteristics of reference blood flow areas stored in the reference information storage 40 are based on. In the present disclosure, a reference blood flow area is defined as a blood flow area already known to be a new blood vessel or a pre-existing blood vessel. As such, when the reference information storage 40 stores, for each reference blood flow area, a relative characteristic of the reference blood flow area relative to a skin surface, the relative characteristic obtainer 34 calculates, for each blood flow area, a relative characteristic of the blood flow area relative to a skin surface specified by the body tissue specifier 31. Alternatively, when the reference information storage 40 stores, for each reference blood flow area, a relative characteristic of the reference blood flow area relative to a bone surface nearest the reference blood flow area, the relative characteristic obtainer 34 calculates, for each blood flow area, a relative characteristic of the blood flow area relative to a bone surface nearest the blood flow area specified by the body tissue specifier 31. The relative characteristic obtainer 34 outputs the relative characteristics of the blood flow areas to the blood flow evaluator 50.

The reference information storage 40 stores one or more reference blood flow areas and, for each reference blood flow area, data of a combination of the following four types of information: (i) information indicating whether the reference blood flow area is a short-axis cross-section of a blood vessel or a long-axis cross-section of a blood vessel; (ii) an individual characteristic of the reference blood flow area; (iii) a relative characteristic of the reference blood flow area; and (iv) information indicating whether the reference blood flow area is a new blood vessel. In the present disclosure, a short-axis cross-section of a blood vessel is a cross-section of the blood vessel taken along a plane crossing the direction in which the blood vessel extends, and a long-axis cross-section of a blood vessel is a cross-section of the blood vessel taken along a plane including the direction in which the blood vessel extends. The reference information storage 40 is implemented by using a storage medium such as, for example, a RAM, a flash memory, a hard disk, or an optical disc. Alternatively, the reference information storage 40 may be integrated into another one component of the image processor 10, and for example, may be integrated into the blood flow evaluator 50. Alternatively, the reference information storage 40 may be external to the image processor 10, in which case the reference information storage 40 may be connected to the image processor 10 via an interface such as USB, eSATA, or SDIO, or may be a device, such as a file server or a Network-attached storage (NAS), accessible from the image processor 10 over a network.

The blood flow evaluator 50, for each blood flow area specified by the blood flow are specifier 32, performs an evaluation of the likelihood of the blood flow area indicating a new blood vessel by using a combination of an individual characteristic of the blood flow area and a relative characteristic of the blood flow area, both obtained by the characteristic obtainer 30. In the following, the evaluation of the likelihood of a blood flow area indicating a new blood vessel may be referred to as likelihood evaluation of a blood flow area. Specifically, the blood flow evaluator 50 compares the combination of characteristics of a processing-target blood flow area calculated by the characteristic obtainer 30 with combinations of characteristics of reference blood flow areas stored in the reference information storage 40. Thus, the blood flow evaluator 50 evaluates whether the combination of characteristics of the processing-target blood flow area are similar to combinations of characteristics of reference blood flow areas known to be new blood vessels or to combinations of characteristics of reference blood flow areas known to be pre-existing blood vessels. The blood flow evaluator 50 includes: a short-axis evaluator 51; a long-axis evaluator 52; and a total evaluator 53. The short-axis evaluator 51 supposes that the processing-target blood flow area is a short-axis cross-section of a blood vessel, and evaluates the processing-target blood flow area by reading out, from the reference information storage 40, data for reference blood flow areas that are short-axis cross-sections of blood vessels. Meanwhile, the long-axis evaluator 52 supposes that the processing-target blood flow area is a long-axis cross-section of a blood vessel, and evaluates the processing-target blood flow area by reading out, from the reference information storage 40, data for reference blood flow areas that are long-axis cross-sections of blood vessels. The total evaluator 53 selects the more reliable one of the evaluation result of the short-axis evaluator 51 and the evaluation result of the long-axis evaluator 52, and outputs the selected evaluation result.

Note that among the components of the image processor 10, the image obtainer 20, the characteristic obtainer 30, and the blood flow evaluator 50 are each implemented by using, for example, hardware such as a Field Programmable Gate Array (FPGA) or an Application Specific Integrated Circuit (ASIC). Further, two or more among the image obtainer 20, the characteristic obtainer 30, and the blood flow evaluator 50 may be integrated into a single hardware component. Further, a part or the entirety of each of the image obtainer 20, the characteristic obtainer 30, and the blood flow evaluator 50 may be implemented by using a single FPGA or a single ASIC. For example, the body tissue specifier 31 and the blood flow area specifier 32 may each be implemented by using a single FPGA. Alternatively, each of the image obtainer 20, the characteristic obtainer 30, and the blood flow evaluator 50 may be individually implemented by using a combination of a memory, a programmable device such as a Central Processing Unit (CPU) or a General Purpose Graphic Processing Unit (GPGPU), and software, or two or more of the image obtainer 20, the characteristic obtainer 30, and the blood flow evaluator 50 may be collectively implemented by using a combination of a memory, a programmable device, and software.

### <Operations>

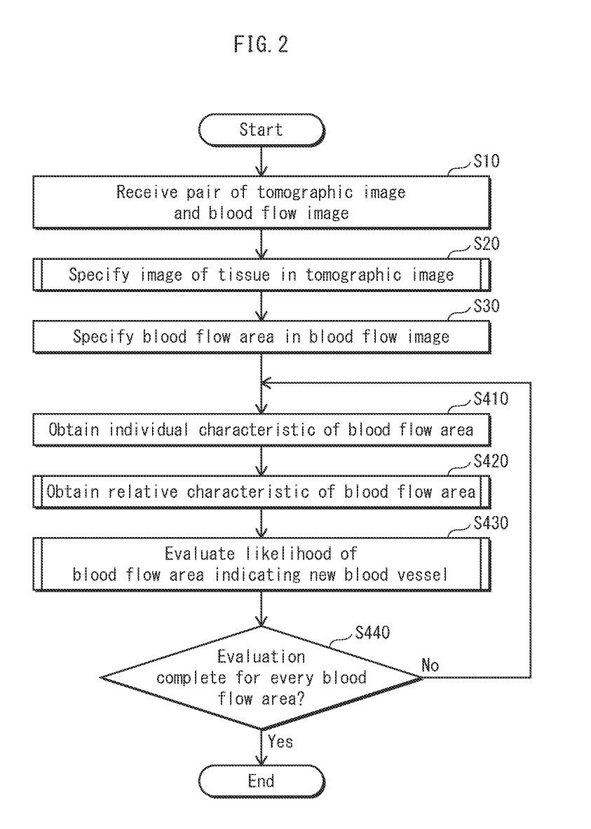

The following describes the operations of the image processor 10. FIG. 2 is a flowchart illustrating the operations of the image processor 10.

First, the image obtainer 20 receives a pair of a blood flow image and a tomographic image (Step S10). Here, when the blood flow image is a moving picture, the image obtainer 20 extracts one frame of the moving picture, and similarly, when the tomographic image is a moving picture, the image obtainer 20 extracts one frame of the moving picture. Further, when the blood flow image and the tomographic image do not share the same coordinate system, the image obtainer 20 performs coordinate conversion on at least one of the blood flow image and the tomographic image so that, after the conversion, the coordinate system of the blood flow image and the coordinate system of the tomographic image match. The image obtainer 20 outputs the pair of the blood flow image and the tomographic image to the characteristic obtainer 30, with the blood flow image and the tomographic image sharing the same coordinate system in any case at this point.

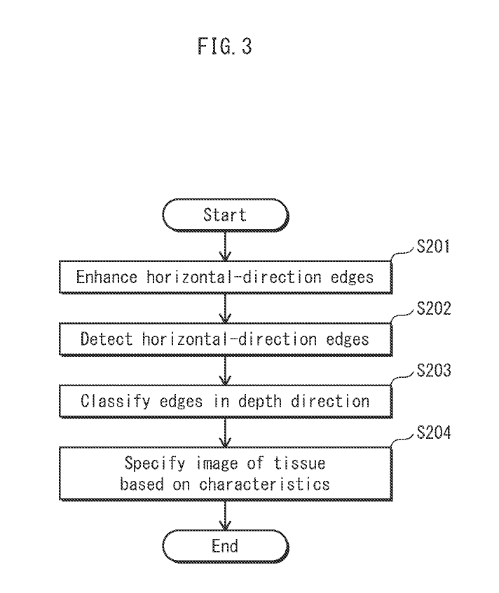

Subsequently, the body tissue specifier 31 specifies images of body tissues in the tomographic image (Step S20). The following describes the details of the processing in Step S20 based on FIG. 3, which is a flowchart illustrating the body tissue specification.

First, the body tissue specifier 31 performs edge enhancement on the tomographic image (Step S201). Here, for example, the body tissue specifier 31 extracts edges from the tomographic image by using a Sobel operator, which extracts edges along a direction perpendicular to the depth direction (horizontal direction), and overlays the extracted edges onto the tomographic image to enhance the horizontal-direction edges.

Then, the body tissue specifier 31 performs edge detection (Step S202). Here, the body tissue specifier 31 may perform the edge detection by performing dynamic programming for example. Specifically, when for example referring to the depth direction and the horizontal direction of the tomographic image as a Y direction and an X direction, respectively, the body tissue specifier 31 extracts, from among all coordinate points having an X coordinate of 1, one or more coordinate points corresponding to pixels with extreme luminance levels (local luminance peaks), and extracts such coordinate points. Then, for each of the extracted coordinate points, the body tissue specifier 31 searches for one or more corresponding points among all coordinates points with X coordinates of 2 by setting the extracted coordinate point as the search start point. Here, a coordinate point that satisfies both the following conditions (i) and (ii) is specified as a corresponding point: (i) the coordinate point corresponds to a pixel having a great luminance level; (ii) a difference (depth difference) between the Y coordinate of the coordinate point and the Y coordinate of the search start point is small. Specifically, in the present embodiment, a coordinate point for which V=(p×yd)+(q×L) is smallest is specified as the corresponding point, where yd denotes the difference between the Y coordinates of the search start point and the coordinate point, L denotes the luminance level of the coordinate point, and coefficients p and q are variables satisfying p>0>q. Here, coordinate points whose values yd are greater than or equal to a predetermined threshold are excluded from the search of the corresponding point. The body tissue specifier 31 sets the corresponding point specified in such a manner as the next search start point. Further, the body tissue specifier 31 specifies, from among all coordinate points having an X coordinate of 2, one or more coordinate points corresponding to pixels with extreme luminance levels (local luminance peaks), extracts such coordinate points, and sets each of such coordinate points as a new search start point. Subsequently, for each search start point with an X coordinate of 2, the body tissue specifier 31 searches for one or more corresponding points among all coordinates points with X coordinates of 3. By repeating such processing and linking search start points with corresponding points, the body tissue specifier 31 detects horizontal-direction edges in the tomographic image.

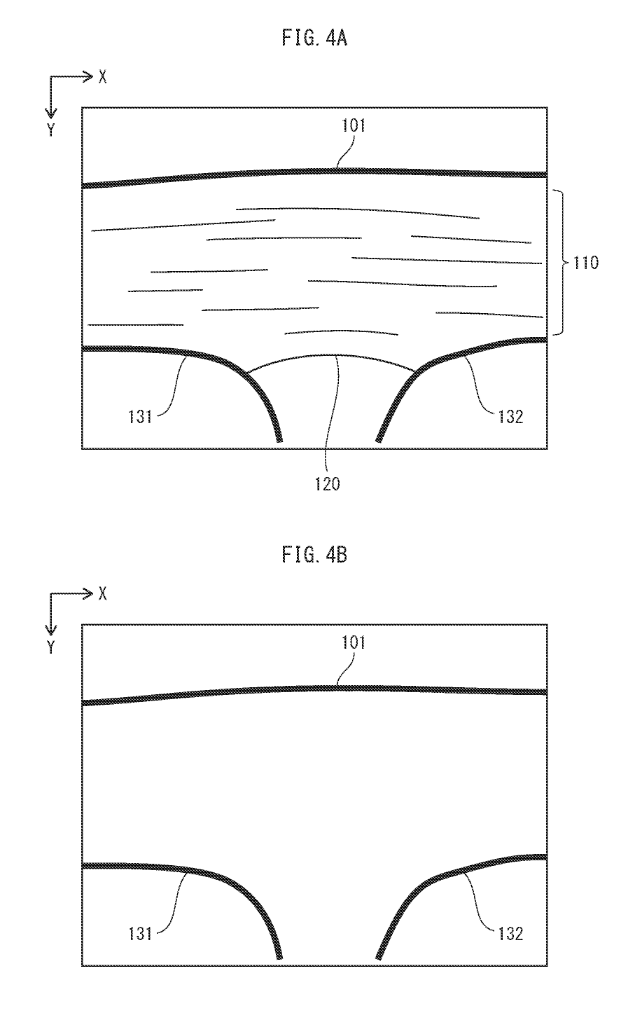

Then, the body tissue specifier 31 classifies the edges detected in the tomographic image by applying an anatomical viewpoint (Step S203), and thereby specifies images of body tissues in the tomographic image (Step S204). The following describes such processing based on edges detected in a tomographic image of a joint. FIG. 4A is a schematic illustrating edges detected in a tomographic image of a joint. The body tissue specifier 31 processes the detected edges in the order they are located in the depth direction from shallow to deep (in order from edges with small Y coordinate values to edges with great Y coordinate values, or that is, in order from high to low in FIG. 4A). Specifically, the body tissue specifier 31 judges whether or not the processing-target edge extends from the left end of the tomographic image to the right end of the tomographic image. When the result of this judgment is affirmative, the body tissue specifier 31 specifies that the processing-target edge is an image of the skin surface. Thus, the body tissue specifier 31 specifies edge 101 in FIG. 4A as a skin surface image. Subsequently, the body tissue specifier 31 continues processing the edges located deeper than the one specified as the skin surface image in the order they are located in the depth direction from deep to shallow, and searches for a pair of edges satisfying the two following conditions: (i) opposing portions of the edges extend downwards in the depth direction; (ii) areas located below the edges in the depth direction have low luminance levels. Thus, the body tissue specifier 31 specifies edges 131 and 132 in FIG. 4A as bone surface images. FIG. 4B illustrates the edges specifies as the skin surface and the bone surfaces. Note that in edge 120 in FIG. 4A is a joint capsule image, and edges in group 110 are tendon images and muscle images.

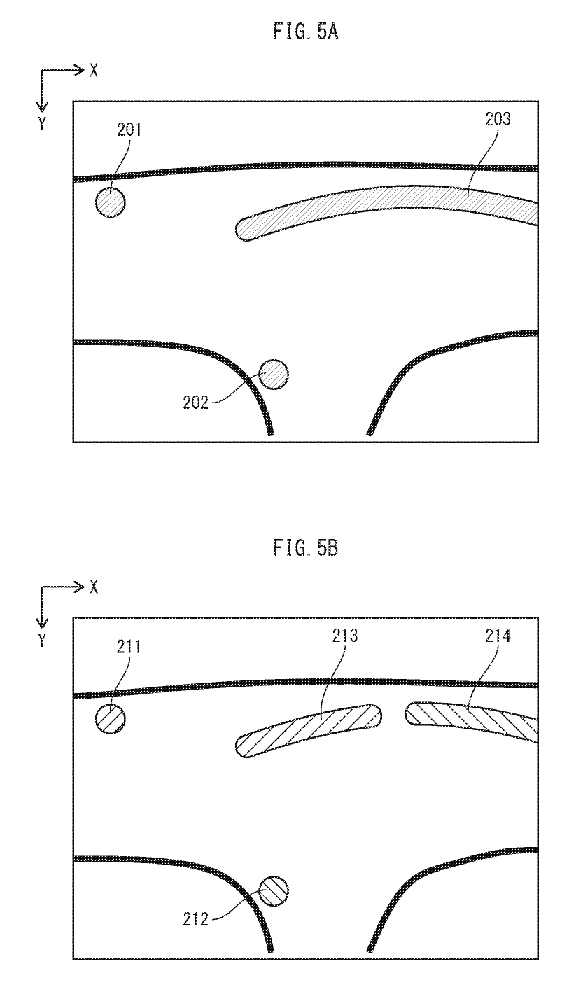

Description continues referring to FIG. 2 once again. Subsequently, the blood flow area specifier 32 specifies one or more blood flow areas in the blood flow image (Step S30). FIG. 5A is a schematic illustrating a power Doppler image serving as the blood flow image. Note that FIG. 5A illustrates a power Doppler image with body tissue images overlaid thereon. A power Doppler image shows blood flow power (i.e., blood flow amount) with color. For example, an area with great blood flow amount may be shown in bright yellow, and an area with small blood flow amount may be shown in dark orange. The blood flow area specifier 32 extracts colored areas from the blood flow image irrespective of color, and specifies each of the extracted areas as a blood flow area. Thus, the blood flow area specifier 32 specifies blood flow areas 201, 202, and 203 in FIG. 5A. Note that when the blood flow image is a contrast medium tomographic image (a CT image), the blood flow area specifier 32 similarly extracts dark contrast medium images from the blood flow image, and specifies each of such contrast medium images as a blood flow area. Meanwhile, FIG. 5B is a schematic illustrating a color Doppler image serving as the blood flow image. Note that FIG. 5B also illustrates a color Doppler image with tissues images overlaid thereon. A color Doppler image shows blood flow direction (i.e., whether blood flow is that towards the ultrasound probe or that away from the ultrasound probe) with color hue and blood flow velocity with color brightness. For example, blood flow towards the ultrasound probe may be shown in red and blood flow away from the ultrasound probe may be shown in blue, and further, blood flow velocity may be shown with higher brightness for higher velocity. Similar to the above-described case where the blood flow image is a color Doppler image, the blood flow area specifier 32 extracts colored areas from the blood flow image irrespective of color, and specifies each of the extracted areas as a blood flow area. Thus, the blood flow area specifier 32 specifies blood flow areas 211, 212, 213, and 214 in FIG. 5B.

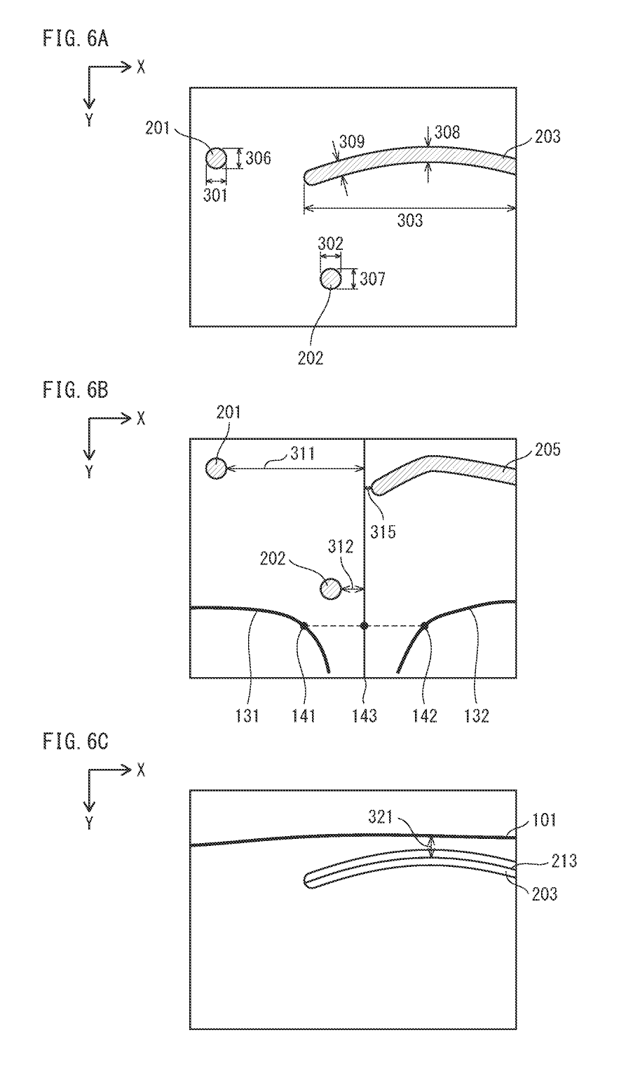

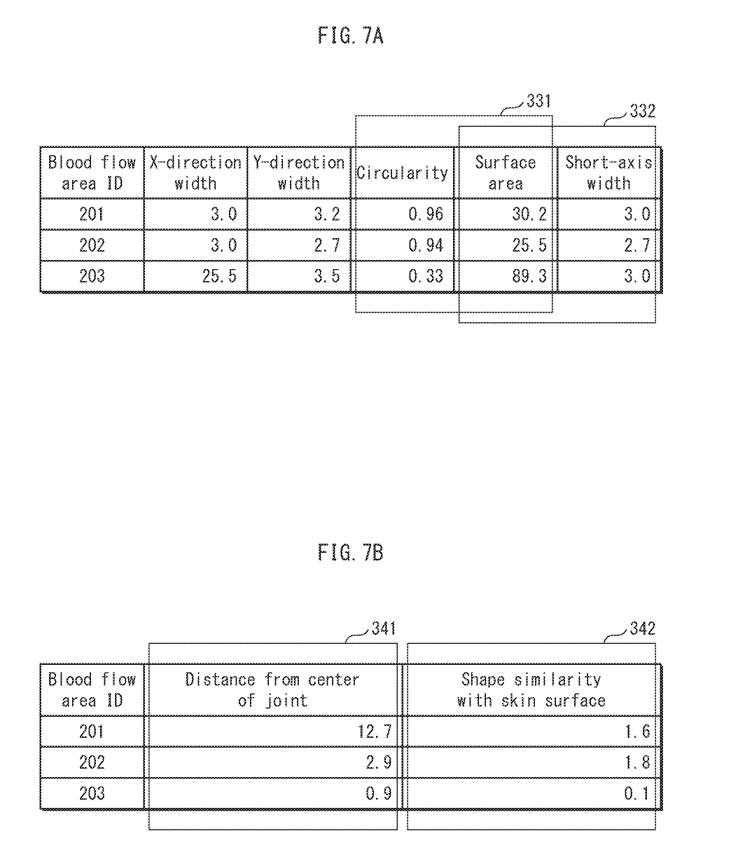

Subsequently, for each blood flow area specified in the blood flow image, the individual characteristic obtainer 33 obtains an individual characteristic indicating the shape of the blood flow area (Step S410). Specifically, for each of the blood flow areas 201, 202, and 203 illustrated in FIG. 6A, the individual characteristic obtainer 33 obtains, as the individual characteristic of the blood flow area, a combination of a surface area, widths, and a circularity of the blood flow area. Specifically, the individual characteristic obtainer 33 obtains the X-direction width 301 and the Y-direction width 306 of the blood flow area 201, the X-direction width 302 and the Y-direction width 307 of the blood flow area 202, and X-direction width 303 and the Y-direction width 308 of the blood flow area 203. Further, the individual characteristic obtainer 33 obtains the circularity of each blood flow area by calculating (surface area×4π)/(perimeter)². The greater the circularity, the closer the blood flow area is to a full circle, whose circularity is 1. Alternatively, instead of obtaining the circularity of each blood flow area, the individual characteristic obtainer 33 may obtain the oblateness of each blood flow, by calculating (Y-direction width)/(X-direction width). Further, the individual characteristic obtainer 33 may only obtain the Y-direction width of each blood flow area. Alternatively, the individual characteristic obtainer 33 may suppose that each blood flow area is a long-axis cross-section of a blood vessel and detect the direction in which the blood vessel extends to obtain a width of the blood flow area in a direction perpendicular to the direction in which the blood vessel extends. In this case, for example, the individual characteristic obtainer 33 may obtain width 309 in FIG. 6A for blood flow area 203. FIG. 7A illustrates examples of the individual characteristics of the detected blood flow areas 201, 202, and 203. Note that individual characteristics 331 in FIG. 7A are to be used by the short-axis evaluator 51, whereas individual characteristics 332 are to be used by the long-axis evaluator 52.

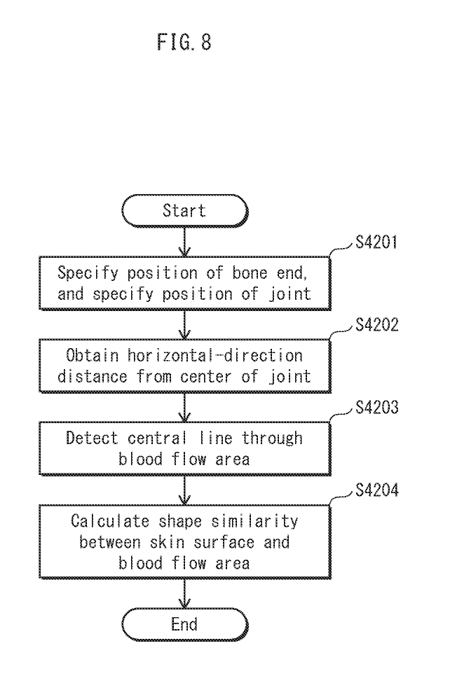

Subsequently, for each blood flow area specified in the blood flow image, the relative characteristic obtainer 34 obtains a relative characteristic indicating the position of the blood flow area relative to a body tissue (Step S420). Specifically, for each blood flow area, the relative characteristic obtainer 34 obtains, as the relative characteristic, a horizontal-direction distance of the blood flow area from the center of the joint in the tomographic image and shape similarity between the blood flow area and a bone surface or a skin surface specified by the body tissue specifier 31. The following describes the details of the processing in Step S420 with reference to FIG. 8, which is a flowchart illustrating the obtaining of relative characteristics.

First, the relative characteristic obtainer 34 specifies the center of the joint in the tomographic image (S4201). Here, the relative characteristic obtainer 34 specifies the center of the joint based on the bone surfaces 131 and 132 specified by the body tissue specifier 31, as illustrated in FIG. 6B. Specifically, the relative characteristic obtainer 34 calculates, for each of the bone surfaces 131 and 132, the curvature of the bone surface after removing minor ups and downs of the bone surface. Subsequently, the relative characteristic obtainer 34 specifies, for each bone surface, a turning point where the curvature becomes positive from negative and where the bone surface is convex, as a bone end of the bone surface. Thus, the relative characteristic obtainer 34 specifies bone ends 141 and 142 of the bone surfaces 131 and 132 in FIG. 6B, respectively. Further, the relative characteristic obtainer 34 detects a bisector 143 of the linear segment connecting the two bone ends 141, 142 as a central line through the joint.

Subsequently, for each blood flow area, the relative characteristic obtainer 34 obtains the horizontal-direction distance between the blood flow area and the central line through the joint as a horizontal-direction distance from the center of the joint (Step S4202). For example, the distance of blood flow area 201 from the central line through the joint is distance 311, the distance of blood flow area 202 from the central line through the joint is distance 312, and the distance of blood flow area 203 from the central line through the joint is distance 315. Note that instead of using the above-described bisector as the central line through the joint, the relative characteristic obtainer 34 may use a straight line that passes through the midpoint of the linear segment connecting the bone ends 141, 142 and that is parallel to the Y-axis as the central line through the joint. Further, when making this modification, the relative characteristic obtainer 34 may obtain, as the horizontal-direction distance of each blood flow area from the center of the joint, an absolute value of a difference between the X coordinate of a representative coordinate point inside the blood flow area and the X coordinate of the central line through the joint. The representative coordinate point is, for example, the center of the blood flow area.

The following describes the shape similarity between a blood flow area and a skin surface, with reference to FIG. 6C. For example, in order to obtain the shape similarity between the blood flow area 203 and the skin surface 101, the relative characteristic obtainer 34 first calculates a central line 213 through the blood flow area 203 (Step S4203). For example, the relative characteristic obtainer 34 calculates the central line 213 by performing thinning on the blood flow area 203.

Then, the relative characteristic obtainer 34 normalizes a change rate of distance 321 between the blood flow area 203 and the skin surface 101, and sets the normalized value as the shape similarity between the blood flow area 203 and the skin surface 101 (Step S4204). Specifically, the relative characteristic obtainer 34 may perform the calculation of the shape similarity according to the following method. In this calculation, y=skin(x) and y=vascular(x) are used as functions representing the skin surface 101 and the blood flow area 203 in the tomographic image, respectively. Further, the change rate of the skin surface 101 (y=skin(x)) and the change rate of the blood flow area 203 (y=vascular(x)) are denoted as sDiff=skin(x+δx)−skin(x) and vDiff=vascular(x+δx)−vascular(x), respectively. In this calculation, the relative characteristic obtainer 34 calculates the shape similarity between the blood flow area 203 and the skin surface 101 by first summing up absolute values of difference sDiff−vDiff over the X coordinate range of the blood flow area 203, and by dividing the sum by the X-direction width of the blood flow area 203 for normalization. Thus, according to this method, the shape similarity between the blood flow area 203 and the skin surface 101 can be expressed as follows.

Σ|sDiff−vDiff|/width [Math. 1]

As such, the more constant the distance between the skin surface 101 and the blood flow area 203 is, or that is, the more similar the shape of the blood flow area 203 is to the shape of the skin surface 101, the smaller the value indicating the shape similarity between the blood flow area 203 and the skin surface 101 is. Further, the value indicating the shape similarity is 0 if the skin surface 101, when moved in the depth direction (in the Y direction), completely matches the central line through the blood flow area 203. Note that instead of calculating the shape similarity between blood flow areas and a skin surface, the relative characteristic obtainer 34 may calculate the shape similarity between blood flow areas and a bone surface. When making this modification, an X coordinate range at which a blood flow area exists but a bone surface does not exist can be simply excluded from the calculation of the shape similarity. For example, when a blood flow area covers an X coordinate range of 30 through 100 and there is one bone surface covering an X coordinate range of 0 through 40 and another bone surface covering an X coordinate range of 60 through 100, the relative characteristic obtainer 34 calculates the shape similarity between the blood flow area and the bone surfaces by first summing up absolute values of difference sDiff−vDiff over the two X coordinate ranges of 30 through 40 and 60 to 100, and by dividing the sum by the X direction width 50 of the blood flow area. FIG. 7B shows examples of relative characteristics of the detected blood flow areas 201, 202, and 203. Note that the relative characteristics 341 in FIG. 7B are to be used by the short-axis evaluator 51, whereas the relative characteristics 342 are to be used by the long-axis evaluator 52.

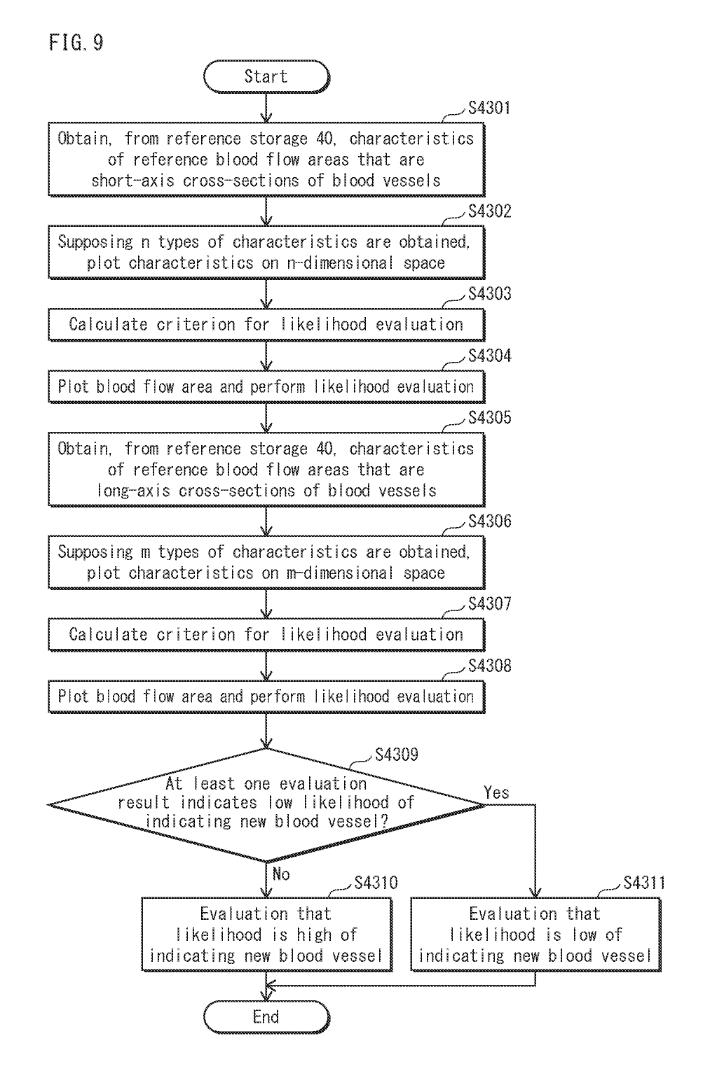

Description continues referring to FIG. 2 once again. Subsequently, the blood flow evaluator 50 performs likelihood evaluation of each blood flow area (Step S430). The blood flow evaluator 50 performs likelihood evaluation of a processing-target blood flow area by comparing the processing-target blood flow area with reference blood flow areas stored in the reference information storage 40. Specifically, the blood flow evaluator 50 performs the likelihood evaluation by performing machine learning, or more specifically, by using a support vector machine, for example. The following description is based on FIG. 9, which is a flowchart illustrating likelihood evaluation of a blood flow area.

First, the short-axis evaluator 51 acquires characteristics of reference blood flow areas that are short-axis cross-sections of blood vessels, from among the characteristics of reference blood flow areas stored in the reference information storage 40 (Step S4301).

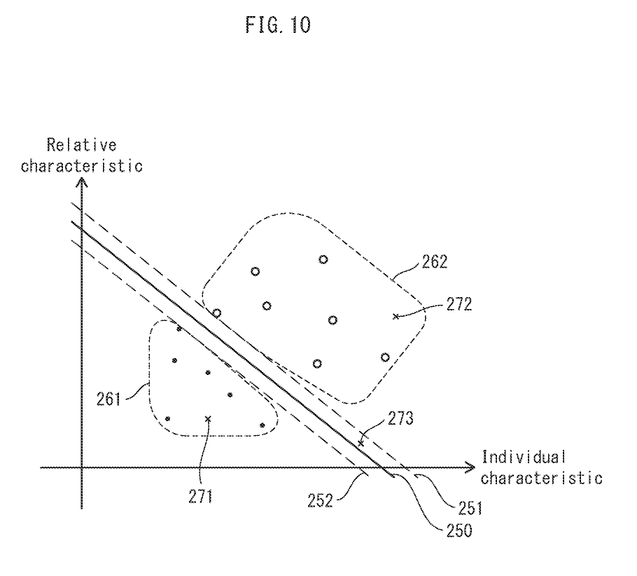

Subsequently, when supposing that the reference information storage 40 stores n types of characteristics for each reference blood flow area that is a short-axis cross-section of a blood vessel, the short-axis evaluator 51 plots the acquired characteristics on an n-dimensional space (Step S4302). Here, since the reference information storage 40 stores, for each reference blood flow area that is a short-axis cross-section of a blood vessel, at least one type of characteristic as an individual characteristic of the reference blood flow area and at least one type of characteristic as a relative characteristic of the reference blood flow area, n is always 2 or greater. Note that the following description is based on an example where n=2, for the sake of simplicity. FIG. 10 is a schematic illustrating the likelihood evaluation using machine learning. FIG. 10 illustrates an example where the likelihood evaluation of a blood flow area is performed by using a surface area of the blood flow area as the individual characteristic of the blood flow area and using the distance from the center of a joint as the relative characteristic of the blood flow area. Thus, in this example, the reference blood flow areas that are short-axis cross-sections of blood vessels, whose characteristics have been acquired, are plotted on a plane defined by a horizontal axis showing surface area and a vertical axis showing distance from the center of a joint. That is, the acquired characteristics of the reference blood flow areas are used as training data in the machine learning. FIG. 10 illustrates reference blood flow areas known to be new blood vessels as block dots, and reference blood flow areas known to be pre-existing blood vessels as outlined circles.

Subsequently, the short-axis evaluator 51 calculates the criterion to be used in the likelihood evaluation (Step S4303). The evaluation criterion is set based on the fact that an area where reference blood flow areas known to be new blood vessels are plotted and an area where reference blood flow area known to be pre-existing blood vessels are plotted indicate different trends. Such difference in trend is a result of the following: (i) individual characteristics of new blood-vessels and individual characteristics of pre-existing blood vessels indicate different trends; (ii) relative characteristics of new blood-vessels and relative characteristics of pre-existing blood vessels indicate different trends; and (iii) the relationship between an individual characteristic and a relative characteristic of a new blood vessel differs from the relationship between an individual characteristic and a relative characteristic of a pre-existing blood vessel. Specifically, reference blood flow areas known to be new blood vessels are plotted all together in one area, and reference blood flow areas known to be pre-existing blood vessels are plotted all together in a different area. For example, in FIG. 10, reference blood flow areas known to be new blood vessels are plotted all together in area 261, and reference blood flow areas known to be pre-existing blood vessels are plotted all together in area 262. As such, the evaluation of the likelihood of a processing-target blood flow area indicating a new blood vessel can be performed according to whether the processing-target blood flow area is plotted in the area where reference blood flow areas known to be new blood vessel are plotted or in the area where reference blood flow areas known to be pre-existing blood vessels are plotted. Specifically, by using the support vector machine, the short-axis evaluator 51 determines the straight line 250 serving as the boundary between the area where reference blood flow areas know to be new blood vessels are plotted and the area where reference blood flow areas known to be pre-existing blood vessels are plotted. Here, the discrimination function y=g(x) (where x denotes individual characteristics and y denotes relative characteristics) for the straight line 250 can be expressed as follows: g(x)=(w×x)+b. In this formula, w and b are set so that the minimum value of |(w×x)+b| is 1. Thus, at least one reference blood vessel known to be a new blood vessel or at least one reference blood vessel known to be a pre-existing blood vessel exists on the broken line 252 expressible as y=g(x)−1. In the example illustrated in FIG. 10, at least one reference blood vessel known to be a new blood vessel exists on the broken line 252. Further, at least one reference blood vessel known to be a new blood vessel or at least one reference blood vessel known to be a pre-existing blood vessel, whichever type of reference blood vessel not existing on the broken line 252, exists on the broken line 251 expressible as y=g(x)+1. In the example illustrated in FIG. 10, at least one reference blood vessel known to be a pre-existing blood vessel exists on the broken line 251. Further, no reference blood flow area exists in the area between the two broken lines 251 and 252. In addition, only reference blood vessels known to be new blood vessels exist in the area expressible as y≦g(x)−1 (the area below the broken line 252), and only reference blood vessel known to be pre-existing blood vessels exist in the area expressible as y≧g(x)+1 (the area above the broken line 251). Note that when n≧3, an (n−1) dimensional boundary is set between reference blood vessels known to be new blood vessels and reference blood vessel known to be pre-existing blood vessels in a similar method. For example, when n=3, the boundary between reference blood vessel known to be new blood vessels and reference blood vessel known to be pre-existing blood vessels would be a plane.

Finally, the short-axis evaluator 51 performs the likelihood evaluation of the processing-target blood flow area based on which area the blood flow area is plotted in. Specifically, the short-axis evaluator 51 evaluates a blood flow area 271 plotted in the area expressed by y≦g(x)−1 as having a high likelihood of indicating a new blood vessel, and that a blood flow area 272 plotted in the area expressed by y=g(x)+1 as having a high likelihood of indicating a pre-existing blood vessel. Meanwhile, the short-axis evaluator 51 determines that a blood flow area 273 that is not plotted in either area and plotted in the area between the two broken lines 251 and 252 cannot be evaluated. Here, note that the likelihood evaluation need not be performed by using the support vector machine, as long as likelihood evaluation can be performed such that blood flow areas having characteristics similar to known pre-existing blood vessels can be evaluated as having a high likelihood of indicating pre-existing blood vessels, and blood flow areas having characteristics similar to known new blood vessels can be evaluated as having a high likelihood of indicating new blood vessels. That is, other machine learning methods are applicable that are capable of evaluating blood flow areas plotted in the area 261 as having a high likelihood of indicating new blood vessels and evaluating blood flow areas plotted in the area 262 as having a high likelihood of indicating pre-existing blood vessels.

Similarly, the long-axis evaluator 52 acquires characteristics of reference blood flow areas that are long-axis cross-sections of blood vessels, from among the characteristics of reference blood flow areas stored in the reference information storage 40 (Step S4305).

Subsequently, when supposing that the reference information storage 40 stores m types of characteristics for each reference blood flow area that is a long-axis cross-section of a blood vessel, the long-axis evaluator 52 plots the acquired characteristics on an m-dimensional space (Step S4306). Here, since the reference information storage 40 stores, for each reference blood flow area that is a long-axis cross-section of a blood vessel, at least one type of characteristic as an individual characteristic of the reference blood flow area and at least one type of characteristic as a relative characteristic of the reference blood flow area, m is always 2 or greater. Note that the following description is based on an example where m=2, for the sake of simplicity. For example, when the likelihood evaluation of a blood flow area is performed by using a width of the blood flow area as the individual characteristic of the blood flow area and using the shape similarity between the blood flow area and a skin surface as the relative characteristic of the blood flow area, the reference blood flow areas whose characteristics have been acquired are plotted on a plane defined by a horizontal axis showing width and a vertical axis showing shape similarity. That is, the acquired characteristics of the reference blood flow areas are used as training data in the machine learning.

Subsequently, the long-axis evaluator 52 calculates the criterion to be used in the likelihood evaluation (Step S4307), and further, performs the likelihood evaluation of the processing-target blood flow area based on the area the blood flow area is plotted in (S4308). Since Steps S4307 and S4308 are similar to Steps S4303 and S4304, respectively, detailed description is not provided of the processing in Steps S4307 and S4308.

Finally, the total evaluator 53 uses the evaluation result of the short-axis evaluator 51 and the evaluation result of the long-axis evaluator 52 for the processing-target blood flow area to make a final evaluation of the likelihood of the blood flow area indicating a new blood vessel (Step S4309). The following describes how the total evaluator 53 performs the final likelihood evaluation. The total evaluator 53 evaluates the processing-target blood flow area to have a low likelihood of indicating a new blood vessel (i.e., to have a high likelihood of indicating a pre-existing blood vessel) (S4311) when at least one of the evaluation result of the short-axis evaluator 51 and the evaluation result of the long-axis evaluator 52 indicates a low likelihood of the blood flow area indicating a new blood vessel, and evaluates the processing-target blood flow area to have a high likelihood of indicating a new blood vessel (Step S4310) in all other cases. The total evaluator 53 performs the final likelihood evaluation in such a manner for the following reasons. Reason (a): When the processing-target blood flow area is a short-axis cross-section of a pre-existing blood vessel, there is a possibility of the evaluation result of the long-axis evaluator 52 incorrectly indicating that the blood flow area has a high likelihood of indicating a new blood vessel, and similarly, when the processing-target blood flow area is a long-axis cross-section of a pre-existing blood vessel, there is a possibility of the evaluation result of the short-axis evaluator 51 incorrectly indicating that the blood flow area has a high likelihood of indicating a new blood vessel. Reason (b): When the processing-target blood flow area is a short-axis cross-section of a new blood vessel, the evaluation result of the long-axis evaluator 52 rarely indicates that the blood flow area has a high likelihood of indicating a pre-existing blood vessel, and similarly, when the processing-target blood flow area is a long-axis cross-section of a new blood vessel, the evaluation result of the short-axis evaluator 51 rarely indicates that the blood flow area has a high likelihood of indicating a pre-existing blood vessel. Reason (c): When the evaluation result of at least one of the short-axis evaluator 51 and the long-axis evaluator 52 indicates that the processing-target blood flow area cannot be evaluated, providing the blood flow area with a final evaluation that likelihood is high of the blood flow area indicating a new blood vessel is beneficial in order to prevent new blood vessels from being overlooked.

When likelihood evaluation of the processing-target blood flow area is completed, the processing in Steps S410 through S430 is executed for another blood flow area if there still exists one or more blood flow areas to be evaluated, whereas processing is terminated if all blood flow areas have been evaluated (Step S440).

### CONCLUSION

The configuration described above, for each blood flow area, evaluates whether the likelihood is high of the blood flow area indicating a new blood vessel by using a combination of an individual characteristic of the blood flow area and a relative characteristic of the blood flow area. As described above, an individual characteristic of a blood flow area is defined as being a characteristic of the blood flow area itself, such as blood flow area shape and/or surface area, whereas a relative characteristic of a blood flow area is defined as a characteristic indicating a relationship between the blood flow area and a body tissue, such as the position of the blood flow area relative to a center of a joint and/or a shape similarity between the blood flow area and a skin surface. Thus, this configuration achieves performing the likelihood evaluation for each blood flow area individually, and further, achieves performing the likelihood evaluation of each blood flow area accurately by using a combination of an individual characteristic of the blood flow area and a relative characteristic of the blood flow area.

### Modification of Embodiment 1

Embodiment 1 describes performing likelihood evaluation of each blood flow area by first separately performing both a primary likelihood evaluation supposing that the blood flow area is a short-axis cross-section of a blood vessel and a primary likelihood evaluation supposing that the blood flow area is a long-axis cross-section of a blood vessel, and then performing a final likelihood evaluation by using the evaluation results of the two primary likelihood evaluations.

Meanwhile, this modification describes a configuration reducing the number of times likelihood evaluation is performed with respect to each blood flow area.

### <Structure>

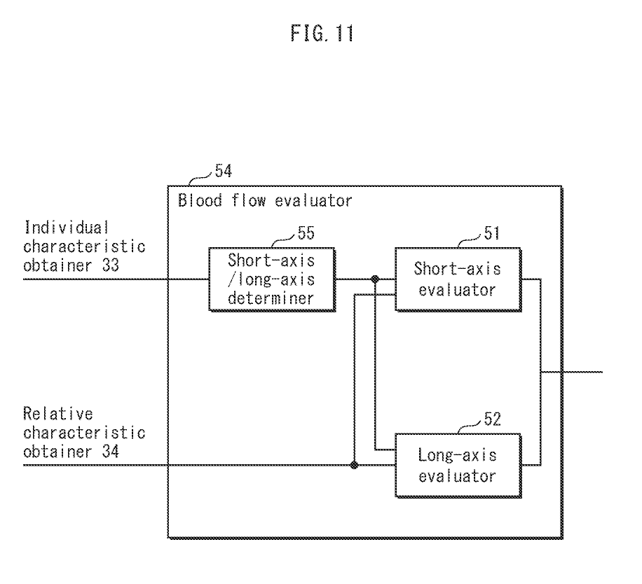

FIG. 11 illustrates functional blocks of a blood flow evaluator 54 pertaining to this modification. Note that in FIG. 11, components already illustrated in FIG. 1 are indicated by the reference signs provided in FIG. 1. Further, the following does not describe such components in detail. The blood flow evaluator 54 includes a short-axis/long-axis determiner 55 in addition to the short-axis evaluator 51 and the long-axis evaluator 52.

The short-axis/long-axis determiner 55, for each blood flow area, performs a determination of whether the blood flow area is a short-axis cross-section of a blood vessel or a long-axis cross-section image of a blood vessel, based on characteristics of the blood flow area. Further, the short-axis/long-axis determiner 55 causes the short-axis evaluator 51 to perform likelihood evaluation of the processing-target blood flow area when determining that the blood flow area is a short-axis cross-section of a blood vessel, and causes the long-axis evaluator 52 to perform likelihood evaluation of the processing-target blood flow area when determining that the blood flow area is a long-axis cross-section of a blood vessel.

### <Operations>

The following describes likelihood evaluation of a blood flow area in this modification. Note that the following does not describe processing other than the likelihood evaluation in detail, since such processing is similar to that in embodiment 1.

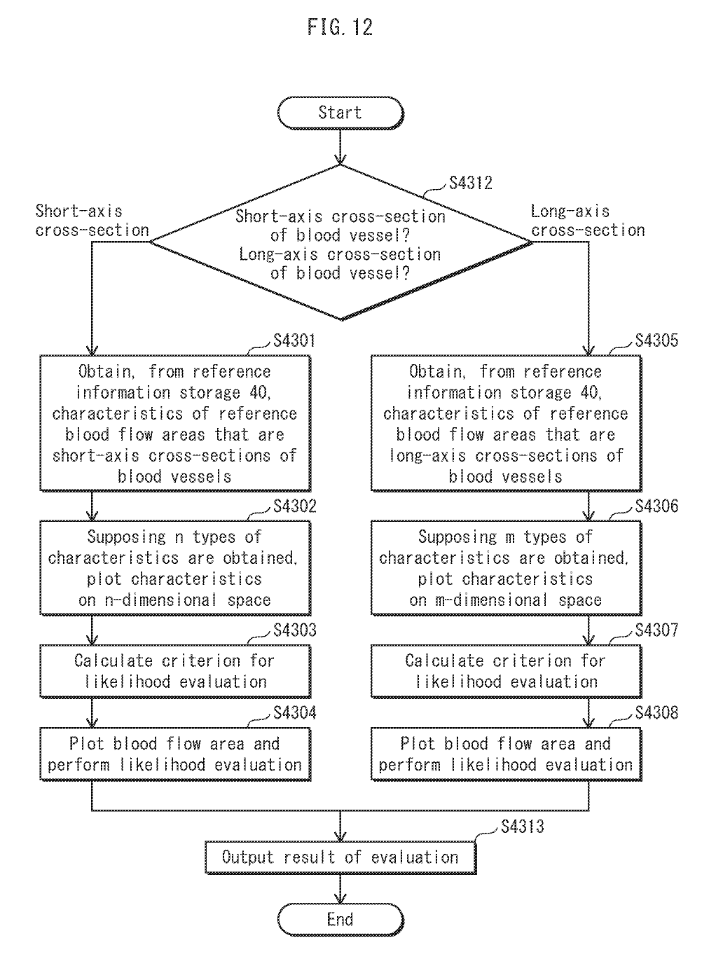

FIG. 12 is a flowchart illustrating the likelihood evaluation in this modification. Note that in FIG. 12, processing steps already illustrated in FIG. 9 are indicated by the step numbers provided in FIG. 9. Further, the following does not describe such processing steps in detail.

First, the short-axis/long-axis determiner 55 determines whether the processing-target blood flow area is a short-axis cross-section of a blood vessel or a long-axis cross-section of a blood vessel, based on characteristics of the blood flow area (Step S4312). For example, the short-axis/long-axis determiner 55 may calculate, for the processing-target blood flow area, a ratio between X-direction and Y-direction widths of the blood flow area (e.g., those illustrated in FIG. 7A), and determine that the processing-target blood flow area is a long-axis cross-section of a blood vessel when the X-direction width is three or more times greater than the Y-direction width. Note that the short-axis/long-axis determiner 55 may make the determination by using any characteristic(s) of the blood flow area, such as the circularity of the blood flow area and/or the surface area of the blood flow area.

The short-axis evaluator 51 acquires characteristics of reference blood flow areas that are short-axis cross-sections of blood vessels, from among the characteristics of reference blood flow areas stored in the reference information storage 40, only when the short-axis/long-axis determiner 55 determines that the processing-target blood flow area is a short-axis cross-section of a blood vessel (Step S4301). Then, the short-axis evaluator 51 plots the acquired characteristics on an n-dimensional space (Step S4302), and calculates the criterion to be used in the likelihood evaluation (Step S4303). Finally, the short-axis evaluator 51 performs the likelihood evaluation of the processing-target blood flow area based on the area the blood flow area is plotted in (S4304).

The long-axis evaluator 52 acquires characteristics of reference blood flow areas that are long-axis cross-sections of blood vessels, from among the characteristics of reference blood flow areas stored in the reference information storage 40, only when the short-axis/long-axis determiner 55 determines that the processing-target blood flow area is a long-axis cross-section of a blood vessel (Step S4305). Then, the long-axis evaluator 52 plots the acquired characteristics on an m-dimensional space (Step S4306), and calculates the criterion to be used in the likelihood evaluation (Step S4307). Finally, the long-axis evaluator 52 performs the likelihood evaluation of the processing-target blood flow area based on the area the blood flow area is plotted in (S4308).

Finally, the blood flow evaluator 54 outputs an evaluation result for the blood flow area (Step S4313). In this modification, only one of the likelihood evaluation in Step S4304 and the likelihood evaluation in Step S4308 is performed for each blood flow area. Thus, the blood flow evaluator 54 outputs the result of whichever evaluation performed, as is.

### CONCLUSION

This structure achieves varying the characteristics and training data used in likelihood evaluation depending upon whether the processing-target blood flow area is a short-axis cross-section of a blood vessel or a long-axis cross-section of a blood vessel. Thus, this structure provides the likelihood evaluation with even higher accuracy, due to evaluation using inappropriate characteristics and inappropriate training data not being performed.

### Embodiment 2

Embodiment 1 and the modification of embodiment 1 described above describe an image processor that performs likelihood evaluation of blood flow areas by using a combination of a blood flow image and a tomographic image that are obtained from an external source.

Meanwhile, the present embodiment describes an ultrasound diagnostic device that generates a blood flow image and a tomographic image through transmission/reception of ultrasound, performs likelihood evaluation of blood flow areas based on the blood flow image and the tomographic image so generated, and outputs an image indicating the result of the likelihood evaluation.

### <Structure>

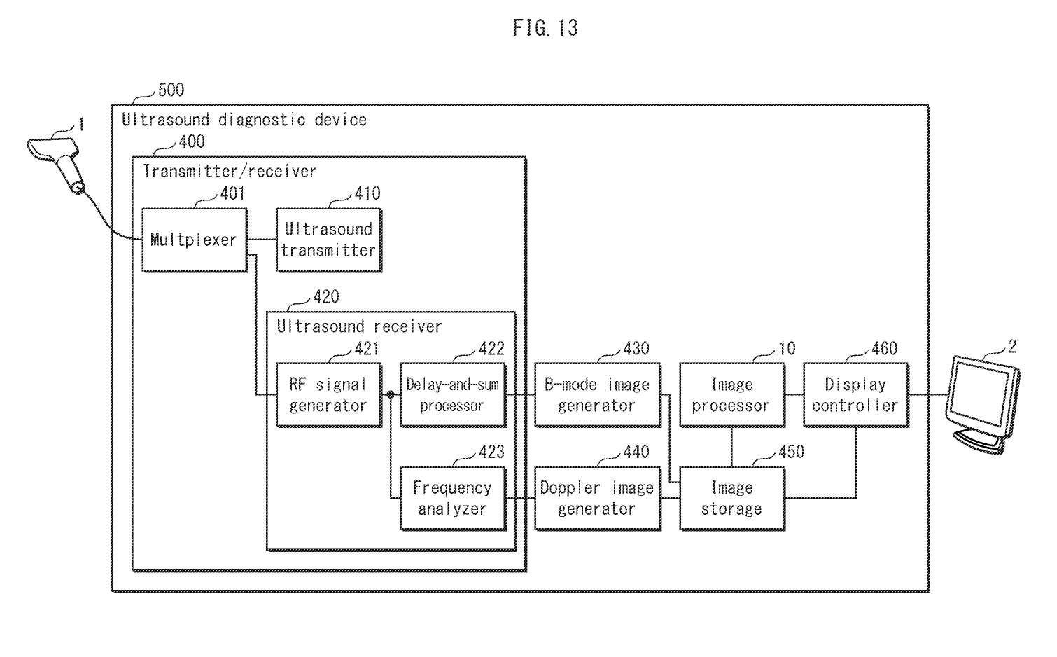

FIG. 13 illustrates functional blocks of an ultrasound diagnostic device 500 pertaining to embodiment 2. The ultrasound diagnostic device 500 includes the image processor 10 pertaining to embodiment 1, and further includes: a transmitter/receiver 400; a B-mode image generator 430; a Doppler image generator 440; an image storage 450; and a display controller 460. The transmitter/receiver 400 includes: a multiplexer 401; an ultrasound transmitter 410; and an ultrasound receiver 420. The ultrasound receiver 420 is a circuit including: an RF signal generator 421; a delay-and-sum processor 422; and a frequency analyzer 423. Further, the transmitter/receiver 400 is connectable with an ultrasound probe 1, and the display controller 460 is connectable with a display 2. FIG. 13 illustrates the ultrasound diagnostic device 500 with the ultrasound probe 1 and the display 2 connected thereto.

The ultrasound probe 1, for example, includes a plurality of undepicted transducer elements arrayed along one direction. Each transducer element may be made, for example, from lead zirconate titanate (PZT). The ultrasound probe 1 is connected to the ultrasound transmitter 410 and the ultrasound receiver 420 via the multiplexer 401. The ultrasound probe 1 receives electric signals (referred to in the following as transmission drive signals) that the ultrasound transmitter 410 generates, and converts the transmission drive signals into ultrasound. With the outer surface thereof where the transducer elements are arrayed in contact with a surface such as a skin surface of the examination subject, the ultrasound probe 1 transmits an ultrasound beam to an examination target inside the examination subject. The ultrasound probe 1 generates the ultrasound beam through conversion of the transmission drive signals, and the ultrasound beam is composed of transmission detection waves transmitted from transducer elements. Further, the ultrasound probe 1 receives reflection detection waves from the examination target as a response to the transmission detection waves. The ultrasound probe 1 converts the reflection detection waves into electric signals (referred to in the following as element reception signals) by using a plurality of transducer elements, and supplies the ultrasound receiver 420 with the element reception signals.

The multiplexer 401 couples the ultrasound transmitter 410 with transducer elements that are selected to drive according to the transmission drive signals. Further, the multiplexer 401 couples the ultrasound receiver 420 with transducer elements that are selected to generate element reception signals.

The ultrasound transmitter 410 generates the transmission drive signals, which are electric signals causing the ultrasound probe 1 to transmit the transmission detection waves. Here, note that the present embodiment mainly describes a case where the ultrasound probe 1 is caused to transmit focused ultrasound. The following describes how acoustic line signals from an entirety of a region of interest (ROI), which is an area corresponding to one ultrasound image, are acquired. In this case, the transmission of transmission detection waves and the reception of reflection detection waves are performed for each transmission focal point, and the transmission focal point is shifted in the direction in which the transducer elements of the ultrasound probe 1 are arrayed. Note that in the following, the term “transmission event” is used to refer to a sequence of one or more ultrasound transmissions performed for transmitting transmission detection waves to the entirety of one ROI. Thus, when causing the ultrasound probe 1 to transmit focused ultrasound, a transmission event for transmitting transmission detection waves to the entirety of one ROI is composed of a plurality of ultrasound transmissions, each of which corresponding to a different transmission focal point. The transmission detection waves to be transmitted to a given transmission focal point are generated by the ultrasound transmitter 410 generating, as the transmission drive signals, pulsar electric signals causing different transducer elements to transmit ultrasound at different timings so that the transmission detection waves transmitted by the transducer elements arrive at the transmission focal point concurrently.

Meanwhile, when a B-mode tomographic image is to be generated, for example, the ultrasound probe 1 may be caused to transmit plane wave ultrasound advancing in a certain direction in place of focused ultrasound. In this case, a transmission event for transmitting transmission detection waves to the entirety of one ROI includes only one ultrasound transmission, and the ultrasound transmitter 410 generates, as the transmission drive signals, pulsar electric signals that cause transducer elements to transmit transmission detection waves concurrently or cause transducer elements to perform ultrasound transmission at different timings such that the ultrasound transmission timing changes at a fixed pitch along the transducer element array and becomes gradually later from one end of the transducer element array to the other end of the transducer element array.

The RF signal generator 421 converts the element reception signals acquired in response to each transmission event into RF signals by performing processing such as amplification and A/D conversion on the element reception signals.

For each transmission event, the delay-and-sum processor 422 specifies, for each measurement point in the ROI, RF signals based on ultrasound reflection from the measurement point, and sums the specified RF signals after performing delaying thereof. Thus, the delay-and-sum processor 422, for each measurement point, generates an acoustic line signal emphasizing ultrasound reflection from the measurement point. Specifically, when focused ultrasound is transmitted, the delay-and-sum processor 422 performs the generation of acoustic line signals in units of sub-areas of the ROI. Here, each sub-area is an area extending in the depth direction, through which transmission detection waves transmitted in one ultrasound transmission propagate, and includes the transmission focal point for the ultrasound transmission and an area around the transmission focal point. Alternatively, when plane wave ultrasound is transmitted, the delay-and-sum processor 422 performs the generation of acoustic line signals for the entirety of the ROI all at once.

The frequency analyzer 423 performs frequency analysis on RF signals acquired in response to a plurality of transmission events, and calculates the average blood flow velocity, blood flow variance, and blood flow power for each measurement point. Specifically, the frequency analyzer 423 first generates complex phase detection signals by performing phase detection, and then performs frequency analysis. Here, phase detection of an RF signal is performed by multiplying the RF signal and a first reference signal having the same frequency as the transmission detection wave, multiplying the RF signal and a second reference signal yielded by performing a 90° phase-shift on the first reference signal, and removing high frequency components by using a low pass filter. Thus, the frequency analyzer 423 generates two complex phase detection signals that are complex conjugates. Subsequently, the frequency analyzer 423 performs processing using a moving target indication (MTI) filter on complex phase detection signals to remove signals corresponding to movements with velocities smaller than or equal to a predetermined velocity, or that is, to remove signals corresponding to movements other than blood flow. For example, signals corresponding to unintended probe movement, etc., are removed. Further, the frequency analyzer 423 for example performs correlation processing with respect to a plurality of complex phase detection signals that correspond to the same transmission focal point and that have been acquired in response to different transmission events. Thus, the frequency analyzer 423 calculates a blood flow power spectrum, and based on the average frequency, the frequency dispersion, and the overall intensity of the blood flow power spectrum, calculates blood flow average velocity, blood flow dispersion, and blood flow power. Note that instead of generating complex phase detection signals as described above, the frequency analyzer 423 may generate complex phase detection signals by performing Fast Fourier Transform. Further, in the above, the frequency analyzer 423 generates complex phase detection signals from RF signals. Alternatively, the frequency analyzer 423 may perform A/D conversion on element reception signals, which are analog signals, during or after the phase detection, or may generate complex phase detection signals based on acoustic line signals, which are generated through delay-and-sum processing.

The B-mode image generator 430 generates B-mode image data corresponding to one frame by performing envelope detection, logarithmic compression, etc., on a plurality of acoustic line signals that the delay-and-sum processor 422 generates based on one transmission event. The B-mode image generator 430 stores the B-mode image data it has generated to the image storage 450.

Meanwhile, the Doppler image generator 440 generates Doppler image data by using the data that the frequency analyzer 423 has calculated for each measurement point. As described above, the frequency analyzer 423 calculates, for each measurement point, blood flow average velocity, blood flow dispersion, and blood flow power. Here, description is provided supposing that the Doppler image generator 440 generates a power Doppler image by using blood flow power at the measurement points. Specifically, the Doppler image generator 440 generates a power Doppler image by converting blood flow power into color. Here, it is preferable that the Doppler image generator 440 express blood flow power by using colors with the same hue or by using colors with similar hues. For example, the Doppler image generator 440 may express points whose blood flow power values are greater than equal to a predetermined value by using bright yellow, points whose blood flow power values are lower than the predetermined value by using dark orange, and points where blood flow power is considered as being zero without any color (transparent). Alternatively, the Doppler image generator 440 may generate a color Doppler image. When generating a color Doppler image, the Doppler image generator 440 creates a color Doppler image by converting blood flow direction into hue and converting average blood flow velocity into brightness. For example, the Doppler image generator 440 may express a point where high-velocity blood flow towards the ultrasound probe 1 is observed with bright red, a point where low-velocity blood flow towards the ultrasound probe 1 is observed with dark red, a point where high-velocity blood flow away from the ultrasound probe 1 is observed with bright blue, a point where low-velocity blood flow away from the ultrasound probe 1 is observed with dark blue, and a point where no blood flow is detected without color (transparent). In any case, the Doppler image generator 440 stores the Doppler image data it has generated to the image storage 450.

The image storage 450 stores B-mode image data generated by the B-mode image generator 430 and Doppler image data generated by the Doppler image generator 440. The image obtainer 20 of the image processor 10 reads out, from the image storage 450, B-mode image data as a tomographic image and Doppler image data as a blood flow image. The image storage 450 is implemented by using a storage medium such as, for example, a RAM, a flash memory, a hard disk, or an optical disc. Note that the image storage 450 and the reference information storage 40 of the image processor 10 may be integrated into a single storage medium.

The display controller 460 reads out B-mode image data and Doppler image data from the image storage 450, performs coordinate conversion onto an orthogonal coordinate system, and causes the display 2 to display the B-mode image data, the Doppler image data, or the B-mode image data with the Doppler image data overlaid thereon. Further, the display controller 460 changes how the Doppler image data is displayed, depending upon the result of the likelihood evaluation by the image processor 10. Specifically, the display controller 460 changes how each blood flow area in the Doppler image data is displayed depending upon whether the result of the likelihood evaluation of the blood flow area by the image processor 10 indicates a high likelihood of indicating a new blood vessel or a high likelihood of indicating a pre-existing blood vessel. For example, the display controller 460 displays a blood flow area evaluated to have a high likelihood of indicating a new blood vessel and a blood flow area evaluated to have a high likelihood of indicating a pre-existing blood vessel by using different colors, by adding emphasis with different effects (e.g., flickering, surrounding with dots and blocks), or by displaying only one of the two different types of blood flow areas.

Note that the multiplexer 401, the ultrasound transmitter 410, the RF signal generator 421, the delay-and-sum processor 422, the frequency analyzer 423, the B-mode image generator 430, the Doppler image generator 440, and the display controller 460 are each implemented by using, for example, hardware such as an FPGA or an ASIC. Further, two or more of among the multiplexer 401, the ultrasound transmitter 410, the RF signal generator 421, the delay-and-sum processor 422, the frequency analyzer 423, the B-mode image generator 430, the Doppler image generator 440, and the display controller 460 may be integrated into a single hardware component. For example, when implementing the RF signal generator 421, the delay-and-sum processor 422, and the frequency analyzer 423 into a single hardware component, the ultrasound receiver 420 would be implemented by using a single hardware component. Further, a part or the entirety of each of the multiplexer 401, the ultrasound transmitter 410, the RF signal generator 421, the delay-and-sum processor 422, the frequency analyzer 423, the B-mode image generator 430, the Doppler image generator 440, and the display controller 460 may be implemented by using a single FPGA or a single ASIC. Alternatively, each of the multiplexer 401, the ultrasound transmitter 410, the RF signal generator 421, the delay-and-sum processor 422, the frequency analyzer 423, the B-mode image generator 430, the Doppler image generator 440, and the display controller 460 may be individually implemented by using a combination of a memory, a programmable device such as a Central Processing Unit (CPU) or a General Purpose Graphic Processing Unit (GPGPU), and software, or two or more of the image obtainer 20, the characteristic obtainer 30, and the blood flow evaluator 50 may be collectively implemented by using a combination of a memory, a programmable device, and software.

### <Operations>

The following describes the operations of the ultrasound diagnostic device 500 pertaining to embodiment 2. FIG. 13 is a flowchart illustrating the operations of the ultrasound diagnostic device 500.

First, the ultrasound diagnostic device 500 sets a ROI inside the examination subject (Step S110). For example, the ultrasound diagnostic device 500 may set the ROI by displaying a tomographic image acquired in advance on the display 2 and receiving specification of the ROI from the examiner when the examiner performs input on an undepicted input unit, such as a touch panel, a mouse, or a track ball. However, the ultrasound diagnostic device 500 need not perform the setting of the ROI in such a manner, and alternatively, for example, the ultrasound diagnostic device 500 may set an entirety of the tomographic image acquired in advance as the ROI, or may set a certain region of the tomographic image acquired in advance including at least the center of the tomographic image as the ROI. Further, the ultrasound diagnostic device 500 may perform the later-described process of acquiring a tomographic image and set the ROI based on the tomographic image so acquired.

Subsequently, the ultrasound diagnostic device 500 performs ultrasound transmission/reception with respect to the ROI by using the ultrasound probe 1, and acquires reception signals from the ROI at the transmitter/receiver 400 (Step S120). In this process, the ultrasound diagnostic device 500 performs one transmission event to acquire reception signals to be used for generating a B-mode image, and in addition, a plurality of transmission events to acquire reception signals to be used for generating a Doppler image. For example, the ultrasound diagnostic device 500 performs a sequence of ten consecutive transmission events to acquire the reception signals to be used for generating the Doppler image. Specifically, the transmitter/receiver 400 first performs the transmission event for the B-mode image and then performs the transmission event sequence for the Doppler image. The transmitter/receiver 400 then outputs RF signals corresponding to the transmission event for the B-mode image to the delay-and-sum processor 422, and outputs RF signals corresponding to the transmission event sequence for the Doppler image to the frequency analyzer 423.

Subsequently, the ultrasound diagnostic device 500 generates the B-mode image (Step S130). Specifically, the delay-and-sum processor 422 generates acoustic line signals by performing delay-and-sum processing on the RF signals acquired from the transmitter/receiver 400, and the B-mode image generator 430 performs processing such as envelope detection and logarithmic compression on the acoustic line images to generate the B-mode image data. The B-mode image generator 430 then stores the B-mode image data to the image storage 450.

Subsequently, the ultrasound diagnostic device 500 generates the Doppler image (Step S140). Specifically, the frequency analyzer 423 performs phase detection and frequency analysis on the RF signals acquired from the transmitter/receiver 400, and the Doppler image generator 440 generates the Doppler image data. The Doppler image generator 440 then stores the Doppler image data to the image storage 450.

Subsequently, the ultrasound diagnostic device 500 performs the likelihood evaluation of blood flow areas (Step S150). Specifically, the image processor 10 obtains the B-mode image data and the Doppler image data from the image storage 450, and performs the likelihood evaluation of blood flow areas by using the B-mode image data as a tomographic image and by using the Doppler image data as a blood flow image. Since the details of the likelihood evaluation of blood flow areas have already been described in embodiment 1, the following does not describe the likelihood evaluation in detail.

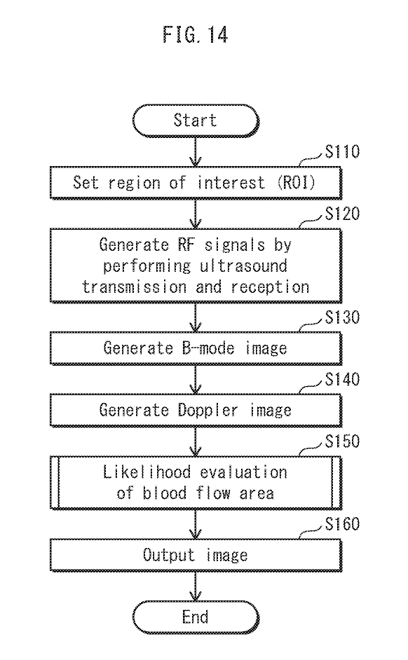

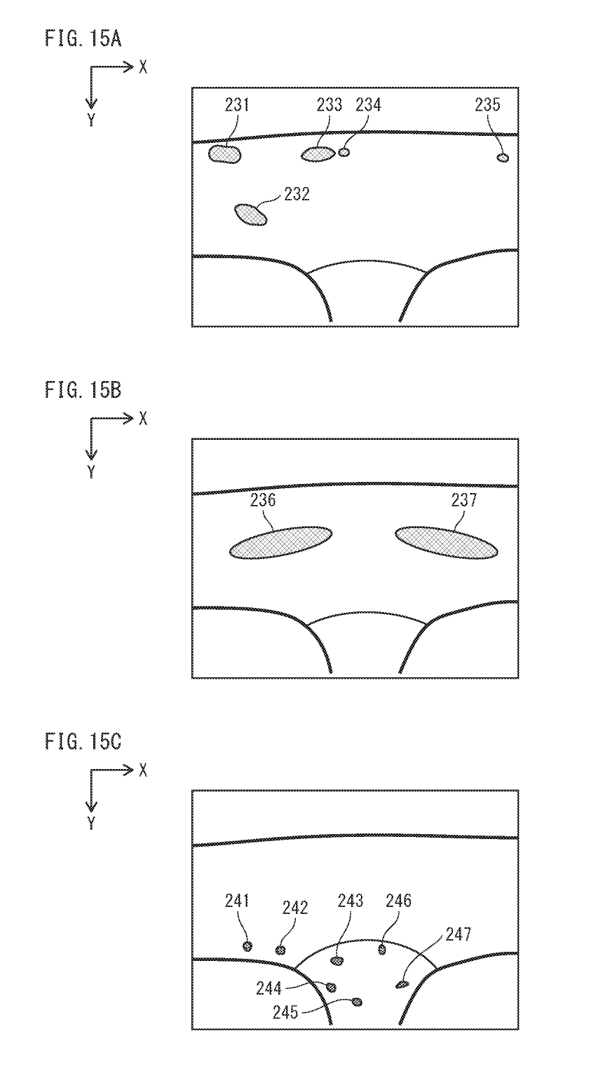

Finally, the ultrasound diagnostic device 500 displays the results of the likelihood evaluation of blood flow areas (Step S160). Specifically, the display controller 460 acquires the B-mode image data and the Doppler image data from the image storage 450. Further, the display controller 460 acquires information indicating the positions of blood flow areas from the blood flow area specifier 32, and acquires the results of the likelihood evaluation of blood flow areas from the blood flow evaluator 50. Further, the display controller 460 causes blood flow areas in the Doppler image data to be displayed differently depending upon the evaluation results of the blood flow areas. For example, FIG. 15A illustrates an example where all blood flow areas have been evaluated as having a high likelihood of indicating a pre-existing blood vessel. In this case, blood flow areas 231 through 235 are all displayed by using a color (e.g., yellow) indicating pre-existing blood vessels. FIG. 15B also illustrates an example where all blood flow areas have been evaluated as having a high likelihood of indicating a pre-existing blood vessel. Thus, blood flow areas 236 and 237 are both displayed by using the color indicating pre-existing blood vessels. Meanwhile, FIG. 14C illustrates an example where all blood flow areas have been evaluated as having a high likelihood of indicating a new blood vessel. In this case, blood flow areas 241 through 247 are all displayed by using a color (e.g., orange) indicating new blood vessels.

### CONCLUSION

The ultrasound diagnostic device 500, with the structure described above, acquires a B-mode image and an ultrasound Doppler image for the same ROI in the examination subject and performs the likelihood evaluation with respect to blood vessels present in the ROI.

### Other Modifications of Embodiments

(1) In embodiments 1 and 2, for each blood flow area, a primary likelihood evaluation supposing that the blood flow area is a short-axis cross-section of a blood vessel and a primary likelihood evaluation supposing that the blood flow area is a long-axis cross-section of a blood vessel are performed in parallel first, and then, a final evaluation of the blood flow area is performed. Meanwhile, in the modification of embodiment 1, for each blood flow area, a determination of whether the blood flow area is a short-axis cross-section of a blood vessel or a long-axis cross-section of a blood vessel is performed first, and then a likelihood evaluation of the blood flow area is performed. However, likelihood evaluation need not be performed in such a manner. For example, the following modification may be made. A long-axis evaluator may be connected in series with the short-axis evaluator 51 to process the output of the short-axis evaluator 51. The long-axis evaluator outputs the evaluation result of the short-axis evaluator 51 as-is when the evaluation result indicates that the likelihood is high of a processing-target blood flow area indicating a pre-existing blood vessel, whereas the long-axis evaluator performs likelihood evaluation of the processing-target blood flow area and outputs the evaluation result only when the evaluation result of the short-axis evaluator 51 indicates that the likelihood is high of the processing-target blood flow area indicating a new blood vessel. Similarly, the following modification may be made. A short-axis evaluator may be connected in series with the long-axis evaluator 52 to process the output of the long-axis evaluator 52. The short-axis evaluator performs likelihood evaluation of a processing-target blood flow area only when the evaluation result of the long-axis evaluator 52 indicates that the likelihood is high of the processing-target blood flow area indicating a new blood vessel.

Alternatively, the following modification may be made. The blood flow area specifier includes a short-axis/long-axis determiner. When the short-axis/long-axis determiner determines that a blood flow area is a short-axis cross-section of a blood vessel, the individual characteristic obtainer and the relative characteristic obtainer obtain only characteristics needed by the short-axis evaluator 51 and output such characteristics only to the short-axis evaluator 51. Meanwhile, when the short-axis/long-axis determiner determines that a blood flow area is a long-axis cross-section of a blood vessel, the individual characteristic obtainer and the relative characteristic obtainer obtain only characteristics needed by the long-axis evaluator 52 and output such characteristics only to the long-axis evaluator 52. This modification reduces processing amount since, when a blood flow area is determined as being a short-axis cross-section of a blood vessel, the obtaining of characteristics appropriate for likelihood evaluation of a blood flow area that is a long-axis cross-section of a blood vessel and likelihood evaluation using such characteristics are not performed, and similarly, when a blood flow area is determined as being a long-axis cross-section of a blood vessel, the obtaining of characteristics appropriate for likelihood evaluation of a blood flow area that is a short-axis cross-section of a blood vessel and likelihood evaluation using such characteristics are not performed.

(2) In embodiments 1 and 2 and the modification of embodiment 1, processing including the obtaining of individual characteristics in Step S410, the obtaining of relative characteristics in Step S420, and the likelihood evaluation in Step S430 is performed one blood flow area at a time. However, such processing may be performed all at once for all blood flow areas specified in Step S30.

(3) In embodiments 1 and 2 and the modification of embodiment 1, the reference information storage 40 stores, for each reference blood flow area, data of a combination of the following four types of information: (i) information indicating whether the reference blood flow area is a short-axis cross-section of a blood vessel or a long-axis cross-section of a blood vessel; (ii) an individual characteristic of the reference blood flow area; (iii) a relative characteristic of the reference blood flow area; and (iv) information indicating whether the reference blood flow area is a new blood vessel. In connection with this, a modification may be made such that each time the blood flow evaluator 50 performs likelihood evaluation of a blood flow area, the result of the likelihood evaluation is additionally stored to the reference information storage 40 as new training data. This modification increases the accuracy of the machine learning. Alternatively, for example, a modification may be made such that each time the blood flow evaluator 50 performs likelihood evaluation of a blood flow area, the result of the likelihood evaluation is additionally stored to the reference information storage 40 as new training data only when the evaluation result is confirmed as being appropriate by a medical practitioner or the like. This modification further increases the accuracy of the machine learning, since only appropriate evaluation results are added to the training data.

(4) In embodiments 1 and 2 and the modification of embodiment 1, in likelihood evaluation supposing that a processing-target blood flow area is a short-axis cross-section of a blood vessel and likelihood evaluation of a processing-target blood flow area that is preemptively determined as being a short-axis cross-section of a blood vessel, circularity and surface area of the blood flow are used as an individual characteristic of the blood flow area and a distance of the blood flow area from a center of a joint is used as a relative characteristic of the blood flow area. Meanwhile, in likelihood evaluation supposing that a processing-target blood flow area is a long-axis cross-section of a blood vessel and likelihood evaluation of a processing-target blood flow area that is preemptively determined as being a long-axis cross-section of a blood vessel, surface area and short-axis width of a blood flow are used as an individual characteristic of the blood flow area and a shape similarity between the blood flow area and a skin surface is used as a relative characteristic of the blood flow area. However, such examples do not limit the individual and relative characteristics that may be used in likelihood evaluation, and likelihood evaluation may be performed by using any characteristic as long as at least one type of information is used as an individual characteristic and at least one type of information is used as a relative characteristic. For example, a modification may be made such that the body tissue specifier 31 further specifies a joint capsule, and likelihood evaluation of a blood flow area is performed by using a position of the blood flow area relative to the joint capsule as a relative characteristic of the blood flow area.

Further, in embodiments 1 and 2 and the modification of embodiment 1, likelihood evaluation is performed based on a pair of a blood flow image and a tomographic image that image a joint. However, likelihood evaluation may be performed based on a pair of a blood flow image and a tomographic image imaging any body part where blood flow is present. When making this modification, the following modifications are to be made in addition: (i) the reference information storage 40 stores reference blood flow areas of the body part imaged in the blood flow image and the tomographic image; (ii) the body tissue specifier 31 extracts a body tissue serving as a reference in the likelihood evaluation, and (iii) the relative characteristic obtainer 34 obtains a relative characteristic of each blood flow area relative to the body tissue serving as a reference.

(5) In embodiment 2, one B-mode image and one ultrasound Doppler image are generated. In connection with this, a modification may be made such that the ultrasound transmitter 410 performs the transmission event for a B-mode image and the transmission sequence for an ultrasound Doppler image one after the other, the B-mode image generator 430 and the Doppler image generator 440 operate one after the other so that a B-mode image and an ultrasound Doppler image are generated one after the other, and the B-mode image and the ultrasound Doppler image so generated are stored to the image storage 450, without the evaluation of blood flow areas being performed.

Further, in the above, the transmission event for a B-mode image and the transmission sequence for an ultrasound Doppler image are performed one after the other. However, for example, a modification may be made such that the ultrasound transmitter 410 performs a sequence of transmission events and outputs RF signals for all transmission events to the Doppler image generator 440 while only outputting RF signals for the final transmission event to the B-mode image generator 430. This modification reduces the number of transmission events that are performed, and increases the frame rates of the B-mode images and the ultrasound Doppler images. Alternatively, a modification may be made such that the transmitter/receiver 400 further includes an RF signal storage and/or an acoustic line signal storage, and the B-mode image generator 430 and/or the Doppler image generator 440 read out necessary signals (RF signals and/or acoustic line signals) from the RF signal storage and/or the acoustic line signal storage.

(6) In embodiment 2, an ultrasound Doppler image is used as a blood flow image. Alternatively, for example, a contrast medium B-mode image, which is generated with an intravascular contrast medium administered to the examination subject, may be obtained and used as a blood flow image.

Further, the image processor 10 may perform likelihood evaluation of blood flow areas by using a pair of any B-mode image stored in the image storage 450 and any ultrasound Doppler image stored in the image storage 450. Alternatively, a modification may be made such that the image processor 10 acquires blood flow images, tomographic images, etc., from one or more external sources, and performs the likelihood evaluation of blood flow areas by using a pair of images such as a pair of a B-mode image and a blood flow image acquired from an external source, a pair of an ultrasound Doppler image and a tomographic image acquired from an external source, or a pair of a blood flow image and a tomographic image both acquired from external source(s).

(7) In embodiment 1 and the modification of embodiment 1, the image obtainer 20 acquires a pair of a blood flow image and a tomographic image imaging the same target region or target regions on the same plane. Alternatively, for example, the image obtainer 20 may acquire, instead of a tomographic image, three-dimensional data such as CT data or MRI data, and information indicating the target region in the blood flow image, and cut out a tomographic image from the three-dimensional data. Similarly, the image obtainer 20 may acquire, instead of a blood flow image, three-dimensional data such as CT data or MRI data, and information indicating the target region in the tomographic image. Alternatively, the image obtainer 20 may acquire both a tomographic image and a blood flow image from three-dimensional data. Specifically, the image obtainer 20 may cut out a tomographic image and a blood flow image from a same plane in the three-dimensional data, or may perform the specification of blood flow areas, the specification of tissue images, the obtaining of the individual and relative characteristics, and the likelihood evaluation of blood flow areas by directly using the three-dimensional data.

(8) The components of any image processor and any ultrasound diagnostic device in the embodiments and the modifications may, in whole or in part, be implemented as an integrated circuit of one chip or a plurality of chips, be implemented as a computer program, and be implemented in any other form. For example, the individual characteristic obtainer and the relative characteristic obtainer may be implemented as one chip, or the blood flow evaluator may be implemented as one chip with the characteristic extractor, etc., implemented as another chip.

In a case in which implementation is achieved by an integrated circuit, implementation may typically be achieved as a large scale integration (LSI). Here, an LSI may variously be referred to as an IC, system LSI, super LSI, or ultra LSI, depending on the degree of integration.

Further, circuit integration methods are not limited to LSI, and implementation may be achieved by a dedicated circuit or general-purpose processor. Alternatively, a field programmable gate array (FPGA) that is programmable after LSI manufacture, and/or reconfigurable processor that allows reconfiguring of connections and settings of internal circuit cells may be used.

Furthermore, if circuit integration technology to replace LSI arises due to progress in semiconductor technology and other derivative technologies, such technology may of course be used to perform integration of functional blocks.

Further, any ultrasound diagnostic device described in the embodiments and the modifications may be implemented by a program stored on a storage medium and a computer that reads and executes the program. The storage medium may be any kind of storage medium, such as a memory card, CD-ROM, etc. Further, the ultrasound diagnostic device pertaining to the present disclosure may be implemented by a program downloadable across a network and a computer that downloads the program across the network and executes the program.

(9) The embodiments described above illustrate specific preferred examples of the technology pertaining to the present disclosure. The values, forms, materials, components, component positions and connections, steps, step order, etc., illustrated in the embodiments represent examples and do not limit the scope of the technology pertaining to the present disclosure. Further, among components in the embodiment, components not disclosed in the independent claims reciting top-level concepts of the technology pertaining to the present disclosure are described as components that may or may not be included, but when included contribute to a preferable form of implementation.

Further, to aid in understanding the technology pertaining to the present disclosure, the scale of components in each drawing referred to in the embodiments may differ from actual implementation. Further, the embodiments do not limit the scope of the technology pertaining to the present disclosure, and instead, various modifications may be made to the embodiments without departing from the scope of the technology pertaining to the present disclosure.

Furthermore, although members such as circuit parts, lead wires, etc., do exist on a substrate of an ultrasound diagnostic device, such electrical wiring and electric circuits may be implemented in various ways based on common knowledge in the technical field, and are therefore not described in detail in the present disclosure as they have no direct relevance to the technology pertaining to the present disclosure. Note that each drawing described above is schematic, and is not an exact representation.

### <<Supplement>>

(1) One aspect of the present disclosure is an image processor including image processing circuitry including: a blood flow image obtainer that obtains a blood flow image in which a blood flow area is mapped, the blood flow area indicating blood flow in an examination subject; a tomographic image obtainer that obtains a tomographic image including an image of a body tissue inside the examination subject; a characteristic obtainer that obtains, based on the obtained blood flow image and the obtained tomographic image, a combination of a first characteristic and a second characteristic of the blood flow area; and a blood flow evaluator that evaluates a likelihood of the blood flow area indicating a new blood vessel, based on the combination of the first characteristic and the second characteristic of the blood flow area, wherein a first characteristic of a blood flow area is defined as being a characteristic of the blood flow area itself, and a second characteristic of a blood flow area is defined as being a characteristic indicating a relationship between the blood flow area and an image of a body tissue in a tomographic image.

Another aspect of the present disclosure is an image processing method including: obtaining a blood flow image in which a blood flow area is mapped, the blood flow area indicating blood flow in an examination subject; obtaining a tomographic image including an image of a body tissue inside the examination subject; obtaining, based on the obtained blood flow image and the obtained tomographic image, a combination of a first characteristic and a second characteristic of the blood flow area; and evaluating a likelihood of the blood flow area indicating a new blood vessel, based on the combination of the first characteristic and the second characteristic of the blood flow area, wherein a first characteristic of a blood flow area is defined as being a characteristic of the blood flow area itself, and a second characteristic of a blood flow area is defined as being a characteristic indicating a relationship between the blood flow area and an image of a body tissue in a tomographic image.

According to this structure, the likelihood evaluation for each blood flow area is performed by using both the first characteristic of the blood flow area and the second characteristic of the blood flow area, and thus, the likelihood evaluation is performed reliably.

(2) In the image processor and the image processing method described in (1) above, the obtained tomographic image may be an image of a joint of the examination subject, and the obtained tomographic image may include, as the image of the body tissue, at least one of an image of a bone surface and an image of a skin surface.

According to this structure, likelihood evaluation of a blood vessel indicated by a blood flow area at a joint region is performed by using, as the second characteristic, information indicating the position of the blood flow area relative to a bone and/or skin.

(3) In the image processor and the image processing method described in (2) above, the obtained tomographic image may at least include an image of a bone surface, the characteristic obtainer may obtain, as the first characteristic of the blood flow area, at least one of a circularity of the blood flow area and a surface area of the blood flow area, and the characteristic obtainer may obtain, as the second characteristic of the blood flow area, at least a horizontal-direction distance between the blood flow area and a center of the joint specified from the image of the bone surface in the obtained tomographic image.

According to this structure, likelihood evaluation of a blood flow area that is a short-axis cross-section of a blood vessel can be performed accurately, by using both the shape of the blood flow area and the position of the blood flow area relative to a position of a joint.

(4) In the image processor and the image processing method described in (2) above, the characteristic obtainer may obtain, as the first characteristic of the blood flow area, at least one of a width of the blood flow area and a surface area of the blood flow area, and the characteristic obtainer may obtain, as the second characteristic of the blood flow area, at least a shape similarity between the blood flow area and an image of a body tissue included in the obtained tomographic image, the body tissue being a bone surface or a skin surface.

According to this structure, likelihood evaluation of a blood flow area that is a long-axis cross-section of a blood vessel can be performed accurately, by using both the shape of the blood flow area and the position of the blood flow area relative to a bone and/or skin.

(5) In the image processor and the image processing method described in (4) above, the shape similarity between the blood flow area and the image of the body tissue included in the obtained tomographic image may indicate a change in depth-direction distance between the blood flow area and the image of the body tissue.

According to this structure, likelihood evaluation is performed accurately based on an anatomical viewpoint.

(6) The image processor and the image processing method described in any one of (1) through (5) may further include a reference information storage storing, for a reference blood flow area, a combination of a first characteristic and a second characteristic of the reference blood flow area, the reference blood flow area being a blood flow area that is already known to be a new blood vessel or a pre-existing blood vessel, and the blood flow evaluator may evaluate the likelihood of the blood flow area indicating a new blood vessel by comparing the combination of the first characteristic and the second characteristic of the blood flow area with the combination of the first characteristic and the second characteristic of the reference blood flow area.

According to this structure, likelihood evaluation of a blood flow area is performed easily and accurately, by comparing the blood flow area with a blood flow area that is already known to be a new blood vessel or a pre-existing blood vessel.

(7) In the image processor and the image processing method described in (6) above, the blood flow evaluator may evaluate the likelihood of the blood flow area indicating a new blood vessel through machine learning using the first characteristic and the second characteristic of the reference blood flow area as training data.

According to this structure, likelihood evaluation of a blood flow area is performed reliably, even when simple computation using a first characteristic and a second characteristic does not suffice to evaluate the blood flow area.

(8) In the image processor and the image processing method described in any one of (1) through (7) above, the obtained tomographic image may be an ultrasound tomographic image, an X-ray tomographic image, or a nuclear magnetic resonance image, and the obtained blood flow image may be an ultrasound Doppler image, a nuclear magnetic resonance image, an ultrasound tomographic image taken with a contrast agent administered to the examination subject, or an X-ray tomographic image taken with a contrast agent administered to the examination subject.

According to this structure, likelihood evaluation is performed by using a pair of a tomographic image and a blood flow image, without limitation on how each of the tomographic image and the blood flow image is produced.

(9) An ultrasound diagnostic device pertaining to an embodiment includes: the image processor described in any one of (1) through (7) above; a transmitter/receiver that causes an ultrasound probe to transmit ultrasound into the examination subject and that receives ultrasound reflection from the examination subject via the ultrasound probe; a B-mode image generator that generates a B-mode image based on the received ultrasound reflection and outputs the generated B-mode image to the tomographic image obtainer as a tomographic image; and a Doppler image generator that generates an ultrasound Doppler image based on a frequency shift in the received ultrasound reflection and outputs the generated ultrasound Doppler image to the blood flow image obtainer as a blood flow image.

This structure achieves generating a B-mode image and an ultrasound Doppler image, and also achieves performing likelihood evaluation of a blood flow area in the ultrasound Doppler image by using a first characteristic indicating a shape of the blood flow area itself and a second characteristic indicating a position of the blood flow area relative to a body tissue.

(10) In the ultrasound diagnostic device described in (9) above, the generated ultrasound Doppler image may be a power Doppler image.

(11) In the ultrasound diagnostic device described in (9) above, the generated ultrasound Doppler image may be a color Doppler image.

These structures achieve visual presentation of blood flow state in the examination subject.

(12) The ultrasound diagnostic device described in any one of (9) through (11) above may further include a display controller that outputs, to a display, an evaluation image that shows, for each blood flow area of the generated ultrasound Doppler image, a result of the evaluation of the likelihood of the blood flow area indicating a new blood vessel.

This structure achieves visual presentation of blood flow state in the examination subject, with each area of blood flow being presented at least with indication of a result of the likelihood evaluation.

(13) In the ultrasound diagnostic device described in (12) above, the evaluation image may show, for each blood flow area of the generated ultrasound Doppler image, the likelihood of the blood flow area indicating a new blood vessel by using color.

This structure achieves an evaluation image that enables visual observation of new blood vessels and pre-existing blood vessels.

(14) In the ultrasound diagnostic device described in any one of (12) and (13) above, the display controller may cause the evaluation image to be displayed overlaid on the generated B-mode image.

This structure achieves an evaluation image that enables visually observing new blood vessels and pre-existing blood vessels in comparison with body tissues.

Although the present invention has been fully described by way of examples with reference to the accompanying drawings, it is to be noted that various changes and modifications will be apparent to those skilled in the art. Therefore, unless such changes and modifications depart from the scope of the present invention, they should be construed as being included therein.

What is claimed is:

1. An image processor comprising
image processing circuitry comprising:
a blood flow image obtainer that obtains a blood flow image in which a blood flow area is mapped, the blood flow area indicating blood flow in an examination subject;
a tomographic image obtainer that obtains a tomographic image including an image of a body tissue inside the examination subject;
a characteristic obtainer that obtains, based on the obtained blood flow image and the obtained tomographic image, a combination of a first characteristic and a second characteristic of the blood flow area; and
a blood flow evaluator that evaluates a likelihood of the blood flow area indicating a new blood vessel, based on the combination of the first characteristic and the second characteristic of the blood flow area, wherein
a first characteristic of a blood flow area is defined as being a characteristic of the blood flow area itself, and a second characteristic of a blood flow area is defined as being a characteristic indicating a relationship between the blood flow area and an image of a body tissue in a tomographic image.

2. The image processor of claim 1, wherein
the obtained tomographic image is an image of a joint of the examination subject, and
the obtained tomographic image includes, as the image of the body tissue, at least one of an image of a bone surface and an image of a skin surface.

3. The image processor of claim 2, wherein
the obtained tomographic image at least includes an image of a bone surface,
the characteristic obtainer obtains, as the first characteristic of the blood flow area, at least one of a circularity of the blood flow area and a surface area of the blood flow area, and
the characteristic obtainer obtains, as the second characteristic of the blood flow area, at least a horizontal-direction distance between the blood flow area and a center of the joint specified from the image of the bone surface in the obtained tomographic image.

4. The image processor of claim 2, wherein
the characteristic obtainer obtains, as the first characteristic of the blood flow area, at least one of a width of the blood flow area and a surface area of the blood flow area, and
the characteristic obtainer obtains, as the second characteristic of the blood flow area, at least a shape similarity between the blood flow area and an image of a body tissue included in the obtained tomographic image, the body tissue being a bone surface or a skin surface.

5. The image processor of claim 4, wherein
the shape similarity between the blood flow area and the image of the body tissue included in the obtained tomographic image indicates a change in depth-direction distance between the blood flow area and the image of the body tissue.

6. The image processor of claim 1 further comprising
a reference information storage storing, for a reference blood flow area, a combination of a first characteristic and a second characteristic of the reference blood flow area, the reference blood flow area being a blood flow area that is already known to be a new blood vessel or a pre-existing blood vessel, wherein
the blood flow evaluator evaluates the likelihood of the blood flow area indicating a new blood vessel by comparing the combination of the first characteristic and the second characteristic of the blood flow area with the combination of the first characteristic and the second characteristic of the reference blood flow area.

7. The image processor of claim 6, wherein
the blood flow evaluator evaluates the likelihood of the blood flow area indicating a new blood vessel through machine learning using the first characteristic and the second characteristic of the reference blood flow area as training data.

8. The image processor of claim 1, wherein
the obtained tomographic image is an ultrasound tomographic image, an X-ray tomographic image, or a nuclear magnetic resonance image, and
the obtained blood flow image is an ultrasound Doppler image, a nuclear magnetic resonance image, an ultrasound tomographic image taken with a contrast agent administered to the examination subject, or an X-ray tomographic image taken with a contrast agent administered to the examination subject.

9. An ultrasound diagnostic device comprising:
the image processor of claim 1;
a transmitter/receiver that causes an ultrasound probe to transmit ultrasound into the examination subject and that receives ultrasound reflection from the examination subject via the ultrasound probe;
a B-mode image generator that generates a B-mode image based on the received ultrasound reflection and outputs the generated B-mode image to the tomographic image obtainer as a tomographic image; and
a Doppler image generator that generates an ultrasound Doppler image based on a frequency shift in the received ultrasound reflection and outputs the generated ultrasound Doppler image to the blood flow image obtainer as a blood flow image.

10. The ultrasound diagnostic device of claim 9, wherein
the generated ultrasound Doppler image is a power Doppler image.

11. The ultrasound diagnostic device of claim 9, wherein
the generated ultrasound Doppler image is a color Doppler image.

12. The ultrasound diagnostic device of claim 9 further comprising
a display controller that outputs, to a display, an evaluation image that shows, for each blood flow area of the generated ultrasound Doppler image, a result of the evaluation of the likelihood of the blood flow area indicating a new blood vessel.

13. The ultrasound diagnostic device of claim 12, wherein
the evaluation image shows, for each blood flow area of the generated ultrasound Doppler image, the likelihood of the blood flow area indicating a new blood vessel by using color.

14. The ultrasound diagnostic device of claim 12, wherein
the display controller causes the evaluation image to be displayed overlaid on the generated B-mode image.

15. An image processing method comprising:
obtaining a blood flow image in which a blood flow area is mapped, the blood flow area indicating blood flow in an examination subject;
obtaining a tomographic image including an image of a body tissue inside the examination subject;
obtaining, based on the obtained blood flow image and the obtained tomographic image, a combination of a first characteristic and a second characteristic of the blood flow area; and
evaluating a likelihood of the blood flow area indicating a new blood vessel, based on the combination of the first characteristic and the second characteristic of the blood flow area, wherein
a first characteristic of a blood flow area is defined as being a characteristic of the blood flow area itself, and a second characteristic of a blood flow area is defined as being a characteristic indicating a relationship between the blood flow area and an image of a body tissue in a tomographic image.

16. The image processing method of claim 15, wherein
the obtained tomographic image is an image of a joint of the examination subject, and
the obtained tomographic image includes, as the image of the body tissue, at least one of an image of a bone surface and an image of a skin surface.

17. The image processing method of claim 16, wherein
the obtained tomographic image at least includes an image of a bone surface,
at least one of a circularity of the blood flow area and a surface area of the blood flow area is obtained as the first characteristic of the blood flow area, and
at least a horizontal-direction distance between the blood flow area and a center of the joint specified from the image of the bone surface in the obtained tomographic image are obtained as the second characteristic of the blood flow area.

18. The image processing method of claim 16, wherein
at least one of a width of the blood flow area and a surface area of the blood flow area is obtained as the first characteristic of the blood flow area, and
at least a shape similarity between the blood flow area and an image of a body tissue included in the obtained tomographic image are obtained as the second characteristic of the blood flow area, the body tissue being a bone surface or a skin surface.
